(19) United States
(12) Patent Application Publication
(10) Pub. No.: US 2025/0146969 A1
(43) Pub. Date: May 8, 2025

(54) MULTI-SENSOR AND EARLY DIAGNOSIS SYSTEM USING SAME

(71) Applicant: INDUSTRY-ACADEMIC COOPERATION FOUNDATION, YONSEI UNIVERSITY, Seoul (KR)

(72) Inventors: Seong Chan JUN, Seoul (KR); Yun Ji HWANG, Seoul (KR); Tae Jong HWANG, Seoul (KR); Jong Woo HONG, Seoul (KR); TIANCI LIU, Seoul (KR)

(21) Appl. No.: 18/933,527

(22) Filed: Oct. 31, 2024

(30) Foreign Application Priority Data
Nov. 3, 2023 (KR) 10-2023-0150567
Nov. 21, 2023 (KR) 10-2023-0162651
Oct. 29, 2024 (KR) 10-2024-0149200

(51) Int. Cl. G01N 27/27; A61B 5/08; G01N 27/327; G01N 33/53; G01N 33/543

(52) U.S. Cl. CPC G01N 27/27; A61B 5/082; G01N 27/3275; G01N 33/5308; G01N 33/5438

(57) ABSTRACT

An embodiment provides a multi-sensor and an early diagnosis system using the same, wherein the multi-sensor diagnoses a disease by using deep learning technology after obtaining a plurality of different electric signals obtained through reactions with target materials that respectively contact a plurality of sensors equipped in the multi-sensor.

### CROSS-REFERENCE TO RELATED APPLICATION

This application claims the benefit of Korean Patent Application No. 10-2023-0150567 filed on Nov. 3, 2023, Korean Patent Application No. 10-2023-0162651 filed on Nov. 21, 2023, and Korean Patent Application No. 10-2024-0149200 filed on Oct. 29, 2024, in the Korean Intellectual Property Office, the disclosure of which are hereby incorporated by reference herein in its entirety.

### BACKGROUND

The disclosure relates to a multi-sensor and an early diagnosis system using the same, and more specifically, to a multi-sensor and an early diagnosis system using the same, wherein the multi-sensor diagnoses a disease by using deep learning technology after obtaining a plurality of different electric signals obtained through reactions with target materials that respectively contact a plurality of sensors equipped in the multi-sensor.

As environmental pollution accelerates and the average life expectancy of humans increases, interest in health is increasing day by day.

In line with the above trend, research and development are actively being conducted to diagnose diseases early with easy methods.

First, among the diseases, dementia, which is a neurological disease, is continuously increasing in number as the average life expectancy of humans increases.

Causes of dementia include degenerative neurological diseases such as Alzheimer's disease and Parkinson's disease, and the proportion of Alzheimer's dementia is the highest.

Research on the causative materials of Alzheimer's disease and Parkinson's disease is currently ongoing, and representative materials have been identified. In the case of Alzheimer's disease, beta amyloid (Amyloid Beta) aggregates to form abnormal brain proteins, and at this time, tau protein is involved in the aggregation of beta amyloid. Parkinson's disease is also caused by a similar algorithm, but the difference is that the protein that aggregates is alpha synuclein, and the aggregation of alpha synuclein involves tau protein in the same way. Therefore, if beta amyloid, tau protein, and alpha synuclein are detected, the possibility of Alzheimer's disease and Parkinson's disease can be predicted. Currently, it is known that brain function abnormalities are examined using methods such as Magnetic Resonance Imaging (MRI) and Positron Emission Tomography (PET) rather than accurate brain protein detection methods.

In relation to this, existing diagnosis of neurodegenerative a disease includes evaluation of psychiatric and behavioral symptoms such as cognitive function, and daily function level, tests for blood that may accompany dementia, and tests for brain function abnormalities through Magnetic Resonance Imaging (MRI) and Positron Emission Tomography (PET). Although research has been conducted to identify the cause material for Alzheimer's disease and Parkinson's disease, it is not being used for testing, and the relatively scientific and accurate MRI and PET tests have the disadvantage of high testing costs.

Next, in order to diagnose a disease related to the human body, such as liver disease, kidney disease, gastric ulcer, duodenal ulcer, lung cancer, and pancreatitis, among the diseases mentioned above, one must visit a hospital and undergo a complex process for a long time.

In relation to this, research is being conducted to detect exhaled gas from the human body and then diagnose a disease related to the human body using gas molecules in the exhaled gas.

Traditional exhaled gas sensors have a metal-based structure, which has the problem of being very vulnerable to environmental factors such as moisture. Deformation due to moisture significantly reduces the accuracy of a sensor, making stable gas measurement difficult.

In addition, the existing mixed gas identification technology was developed using deep learning, but its ability was only to roughly identify the type of gas. This was insufficient to accurately determine the ratio of detailed mixed gas, and there were problems with various restrictions in its use, such as the inability to measure at room temperature.

### RELATED ART DOCUMENT

### Patent Document

(Patent document 1) Republic of Korea Patent Publication No. 10-2418399 (Jul. 4, 2022)

### SUMMARY

An aspect of the disclosure is to provide a multi-sensor and an early diagnosis system using the same, wherein the multi-sensor diagnoses a disease by using deep learning technology after obtaining a plurality of different electric signals obtained through reactions with target materials that respectively contact a plurality of sensors equipped in the multi-sensor.

In addition, an aspect of the disclosure is to provide a multi-sensor and an early diagnosis system using the same, wherein the multi-sensor easily determines the composition of a target material using a portable device and diagnoses a disease based on the composition of the target material.

Specifically, an aspect of the disclosure is to provide a multi-sensor and an early diagnosis system using the same, wherein it is possible to diagnose Alzheimer's disease and Parkinson's disease simultaneously by generating and measuring different reactive signals through an interaction of target materials flowing into nine channels and a sensor portion based on a graphene oxide, and then inputting the obtained results into a deep learning model and performing regression analysis to derive the presence and mixing ratio of alpha-synuclein, beta-amyloid, and tau protein, which are causative materials of degenerative neurological disease.

Specifically, an aspect of the disclosure is to provide a multi-sensor and an early diagnosis system using the same, wherein it is possible to diagnose any one of liver, kidney, gastric ulcer, duodenal ulcer, odor-related disease, lung cancer, and pancreatitis by generating and measuring different reactive signals through an interaction of a sensor portion based on an ssDNA-bound graphene and a single graphene and a target material flowing into nine channels, and then inputting the obtained results into a deep learning model and deriving the composition ratio of a disease biomarker gases contained in an exhaled gas through regression analysis.

The aspect of the disclosure is not limited to that mentioned above, and other aspects not mentioned will be clearly understood by those skilled in the art from the description below.

The disclosure provides a multi-sensor, including: a substrate portion; a sensor portion including a plurality of sensors arranged in a matrix on the substrate portion; a plurality of electrode portions arranged radially from the center of the sensor portion on the substrate portion and electrically connected to the plurality of sensors; and a multi-channel portion positioned above the plurality of sensors, and provided with a plurality of body holes through which target materials are introduced, wherein the plurality of sensors generate different electric signals for the target materials and transmit the different electric signals to the plurality of electrode portions, respectively.

In an embodiment of the disclosure, the target materials, which are liquid protein separated from blood, may include alpha synuclein, beta amyloid, and tau protein.

In an embodiment of the disclosure, the different electric signals may be different electrical reactive signals, and the plurality of sensors may interact with the target materials to generate the different electrical reactive signals for detecting a cause material of degenerative neurological a disease and transmit the different electrical reactive signals to the plurality of electrode portions, respectively.

In an embodiment of the disclosure, the plurality of sensors may be made of different graphene oxides and generate different electrical reactive signals due to changes in the properties of the graphene oxides through interactions with the target materials.

In an embodiment of the disclosure, the target materials, which are an analysis target gas that is an animal's exhaled gas, may include ammonia, hydrogen sulfide, and nitrogen monoxide.

In an embodiment of the disclosure, the different electric signals may be different electrochemical signals, and the plurality of sensors may generate the different electrochemical signals for diagnosing any one of liver, kidney, gastric ulcer, duodenal ulcer, odor-related disease, lung cancer, and pancreatitis, and transmit the different electrochemical signals to the plurality of electrode portions, respectively.

In an embodiment of the disclosure, the plurality of sensors may be an upper sensor formed on the upper surface of the substrate portion and arranged in row 1, a central sensor formed on the upper surface of the substrate portion and arranged in row 2 so as to be positioned below the upper sensor, and a lower sensor formed on the upper surface of the substrate portion and arranged in row 3 so as to be positioned below the central sensor, the upper sensor may be a second reduced graphene oxide, the central sensor may be a first reduced graphene oxide, and the lower sensor may be a graphene oxide.

In an embodiment of the disclosure, an ssDNA-bound graphene may include a graphene functionalized with one type of ssDNA and a graphene functionalized with two types of ssDNA, the plurality of sensors may be an upper sensor formed on the upper surface of the substrate portion and arranged in row 1, a central sensor formed on the upper surface of the substrate portion and arranged in row 2 so as to be positioned below the upper sensor, and a lower sensor formed on the upper surface of the substrate portion and arranged in row 3 so as to be positioned below the central sensor, the upper sensor may be a graphene functionalized with the one type of ssDNA, the central sensor may be a graphene functionalized with the two types of ssDNA, and the lower sensor may be a single graphene.

In addition, the disclosure provides an early diagnosis system using a multi-sensor, the early diagnosis system including: the multi-sensor described above; a measuring device electrically connected to the plurality of electrode portions and measuring the different electric signals; and a diagnostic device that inputs the different electric signals transmitted from the measuring device into a deep learning model and then diagnoses a disease through regression analysis, wherein the diagnostic device includes a deep learning portion that improves a diagnostic speed by only using an encoding scheme that increases and then decreases the number of channels in which the different electrical signals, which are one-dimensional signals, are each grouped.

In an embodiment of the disclosure, the target materials, which are liquid protein separated from blood, may include alpha synuclein, beta amyloid, and tau protein, the different electric signals may be different electrical reactive signals, and the diagnostic device may include a data storage portion that stores the different electrical reactive signals transmitted from the measuring device, and a diagnostic portion that simultaneously diagnoses Alzheimer's disease and Parkinson's disease by determining the presence and mixing state of the alpha synuclein, the beta amyloid, and the tau protein.

In an embodiment of the disclosure, the diagnostic portion may diagnose a patient who provided the target materials as suffering from the Alzheimer's disease when the mixing ratio of the alpha synuclein, the beta amyloid, and the tau protein is 3-5:6-14:2-3.

In an embodiment of the disclosure, the diagnostic portion may diagnose a patient who provided the target materials as suffering from the Parkinson's disease when the mixing ratio of the alpha synuclein, the beta amyloid, and the tau protein is 5-13:4-8:1-3.

In an embodiment of the disclosure, the different electric signals may be different electrochemical signals, the target materials, which are an analysis target gas that is an animal's exhaled gas, may include ammonia, hydrogen sulfide, and carbon monoxide, and the diagnostic device may include a data storage portion that stores the different electrochemical signals transmitted from the measuring device, and diagnostic portion that diagnoses any one of liver disease, kidney disease, gastric ulcer, duodenal ulcer, odor-related disease, lung cancer, and pancreatitis by determining the type and composition ratio of the analysis target gas.

In an embodiment of the disclosure, the diagnostic portion may diagnose as suffering from the liver disease, kidney disease, gastric ulcer and duodenal ulcer when the composition ratio of the ammonia is the highest among the ammonia, hydrogen sulfide and carbon monoxide.

In an embodiment of the disclosure, the diagnostic portion may diagnose as suffering from the odor-related a disease and pancreatitis when the composition ratio of the hydrogen sulfide is the highest among the ammonia, hydrogen sulfide and carbon monoxide.

In an embodiment of the disclosure, the diagnostic portion may diagnose as suffering from lung cancer when the composition ratios of the hydrogen sulfide and the carbon monoxide among the ammonia, the hydrogen sulfide and the carbon monoxide are the same.

An effect of the disclosure according to the above configuration is that it is possible to obtain a plurality of different electric signals through reactions with target materials that respectively contact a plurality of sensors equipped in a multi-sensor, and then it is possible to rapidly diagnose a disease using deep learning technology.

In addition, an effect of the disclosure according to the above configuration is that it is possible to provide convenience to a user by easily determining the composition of a target material using a portable device and diagnosing a disease based on the composition of the target material.

In addition, an effect of the disclosure according to the above configuration is that it is possible to noninvasively diagnose Alzheimer's disease and Parkinson's disease simultaneously by deriving the presence and mixing ratio of alpha synuclein, beta amyloid, and tau protein, which are the cause materials of degenerative neurodegenerative diseases, by measuring different reactive signals through an interaction of a target material and a sensor portion based on a graphene oxide flowing into nine channels, and then inputting the obtained results into a deep learning model and performing regression analysis thereon.

In addition, an effect of the disclosure according to the above configuration is that it is possible to non-invasively diagnose any one of liver, kidney, gastric ulcer, duodenal ulcer, odor-related disease, lung cancer, and pancreatitis by generating and measuring different reactive signals through an interaction of a target material flowing into nine channels and a sensor portion based on an ssDNA-bound graphene and a single graphene, and then inputting the obtained result values into a deep learning model and performing regression analysis thereon.

The effects of the disclosure are not limited to the effects described above, and should be understood to include all effects that are inferable from the configuration of the disclosure described in the detailed description or claims of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view from one direction showing a multi-sensor according to first and second embodiments of the disclosure;

FIG. 2 is a plan view from one direction showing a detailed configuration of a substrate portion, a sensor portion, and an electrode portion provided in a multi-sensor according to first and second embodiments of the disclosure;

FIG. 3 is a perspective view from one direction actually simulating FIG. 2;

FIG. 4 ((a), (b), (c), and (d)) is schematic views showing a process of forming a sensor portion provided in a multi-sensor according to an embodiment of the disclosure;

FIG. 5 is a conceptual view showing an interaction between a target material and a sensor portion provided in a multi-sensor according to a first embodiment of the disclosure;

FIG. 6 is a view showing a graphene functionalized with one type of ssDNA, a graphene functionalized with two types of ssDNA, and a single graphene forming a multi-sensor and an upper sensor, a central sensor, and a lower sensor provided in the multi-sensor according to a second embodiment of the disclosure;

FIG. 7 is a block view showing an early diagnosis system using a multi-sensor according to first and second embodiments of the disclosure;

FIG. 8 is a schematic view showing a process of an early diagnosis system using a multi-sensor according to a first embodiment of the disclosure deriving the presence and mixing ratio of a causative material of a degenerative neurological disease through deep learning;

FIGS. 9A, 9B, 9C, 9D, 9E, 9F, 9G, 9H, and 9I are each a graph showing currents according to voltages for nine channels obtained by measuring different reactive signals by a measuring device equipped in an early diagnosis system using a multi-sensor according to first embodiment of the disclosure;

FIG. 10 is a schematic view showing that a deep learning portion equipped in an early diagnosis system using a multi-sensor according to first and second embodiments of the disclosure calculates and performs deep learning on a one-dimensional signal in an encoding manner;

FIGS. 11A and 11B are each a graph showing the accuracy and mixing ratio according to the ratio of alpha synuclein, beta amyloid, and tau protein using a deep learning model in a deep learning portion of an early diagnosis system using a multi-sensor according to a first embodiment of the disclosure;

FIG. 12C is a photograph showing the collection of a human exhaled gas using a sampling bag (Tedlar Bag) in an early diagnosis system using a multi-sensor according to a second embodiment of the disclosure;

FIG. 12B is a photograph showing the acquisition of different electrochemical signals using a plurality of sensors in an early diagnosis system using a multi-sensor according to a second embodiment of the disclosure;

FIG. 12C is a graph for the reactivity over time obtained in response to a multi-sensor in an early diagnosis system using a multi-sensor according to a second embodiment of the disclosure;

FIG. 13 is a schematic view showing a convolutional neural network (CNN) architecture used for calculating a one-dimensional signal in an encoding manner and deep learning determination in a deep learning portion provided in an early diagnosis system using a multi-sensor according to a second embodiment of the disclosure;

FIG. 14A is a photograph showing wireless communication with a tablet PC, which is a smart device, using a Raspberry Pi in an early diagnosis system using a multi-sensor according to a second embodiment of the disclosure;

FIG. 14B is a photograph showing monitoring of a deep learning determination process of an analysis target gas in an early diagnosis system using a multi-sensor according to a second embodiment of the disclosure;

FIGS. 15A, 15B, 15C, 15D, 15E, 15F, and 15G are each a graph showing data obtained when a deep learning portion of an early diagnosis system using a multi-sensor according to a second embodiment of the disclosure performs deep learning based on different electrochemical signals, which are one-dimensional signals;

FIGS. 16A, 16B, and 16C are each a graph showing the reactivity over time of the composition ratio of an analysis target gas obtained by a multi-sensor equipped in an early diagnosis system using a multi-sensor according to a second embodiment of the disclosure;

FIG. 17 is a graph showing the reactivity over time of the composition ratio of an analysis target gas in an early diagnosis system using a multi-sensor according to a second embodiment of the disclosure;

FIG. 18 ((a) and (b)) is graphs showing the before and after of the augmentation of data obtained when a deep learning portion equipped in an early diagnosis system using a multi-sensor according to a second embodiment of the disclosure performs deep learning based on different electrochemical signals, which are one-dimensional signals;

FIG. 19 is a graph showing the composition ratio of an analysis target gas obtained through deep learning in an early diagnosis system using a multi-sensor according to a second embodiment of the disclosure;

FIGS. 20A, 20B, and 20C are each a graph showing response graphs of an analysis target gas under high-humidity conditions in an early diagnosis system using a multi-sensor according to a second embodiment of the disclosure;

FIG. 20D is a view showing the results of deep learning classification accuracy for the graphs of FIGS. 20A, 20B, and 20C;

FIG. 21A is a graph predicting the composition ratio of NH₃ and H₂S by analyzing different electrochemical signals for an analysis target gas under high-humidity conditions in an early diagnosis system using a multi-sensor according to a second embodiment of the disclosure;

FIG. 21B is a graph predicting the composition ratio of NO and H₂S by analyzing different electrochemical signals for an analysis target gas under high-humidity conditions using deep learning in an early diagnosis system using a multi-sensor according to a second embodiment of the disclosure; and

FIG. 21C is a graph predicting the composition ratio of NO and NH₃ by analyzing different electrochemical signals for an analysis target gas under high-humidity conditions using deep learning in an early diagnosis system using a multi-sensor according to a second embodiment of the disclosure.

### DETAILED DESCRIPTION

Hereinafter, the disclosure will be described with reference to the accompanying drawings. However, the disclosure may be implemented in various different forms, and therefore is not limited to the embodiments described herein. In addition, in order to clearly describe the disclosure in the drawings, parts that are not related to the description are omitted, and similar parts are given similar drawing reference numerals throughout the specification.

In the entire specification, when a part is said to be “connected (linked, contacted, coupled)” to another part, this includes not only the case where it is “directly connected” but also the case where it is “indirectly connected” with another member in between. In addition, when a part is said to “include” a certain component, this does not mean that other components are excluded unless otherwise specifically stated, but that other components may be additionally provided.

The terms used in this specification are used only to describe specific embodiments and are not intended to limit the disclosure. The singular expression includes the plural expression unless the context clearly indicates otherwise. In this specification, the terms “include” or “have” are intended to specify the presence of a feature, number, step, operation, component, part, or combination thereof described in the specification, but should be understood as not excluding in advance the possibility of the presence or addition of one or more other features, numbers, steps, operations, components, parts, or combinations thereof.

Hereinafter, embodiments of the disclosure will be described in detail with reference to the accompanying drawings.

### 1. Multi-Sensor 100—First Embodiment

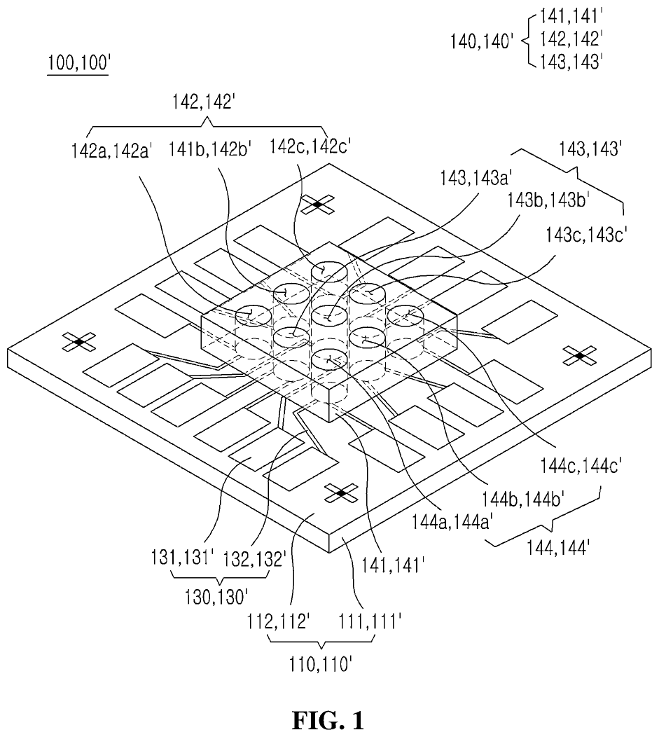

Hereinafter, a multi-sensor according to a first embodiment of the disclosure will be described with reference to FIGS. 1 to 5.

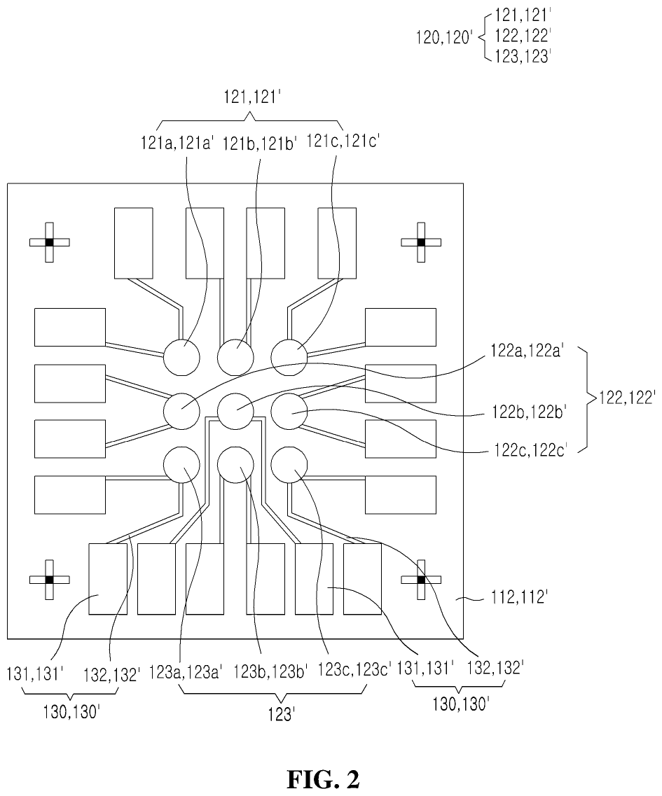

FIG. 1 is a perspective view from one direction showing a multi-sensor according to first and second embodiments of the disclosure. FIG. 2 is a plan view from one direction showing a detailed configuration of a substrate portion, a sensor portion, and an electrode portion provided in a multi-sensor according to first and second embodiments of the disclosure.

Referring to FIGS. 1 and 2, a multi-sensor 100 according to a first embodiment of the disclosure includes a substrate portion 110, a sensor portion 120, electrode portions 130, and a multi-channel portion 140, and may also be implemented as a portable device.

In addition, the multi-sensor 100 according to the first embodiment of the disclosure may be manufactured from various materials including plastic with a width of at least 15 cm, a length of at least 15 cm, and a height of 10 cm.

The substrate portion 110 includes a substrate 111 and a silicon wafer 112.

The substrate 111 has a flat plate shape with a predetermined thickness, and a silicon wafer 112 is coated on the upper surface of the substrate 111.

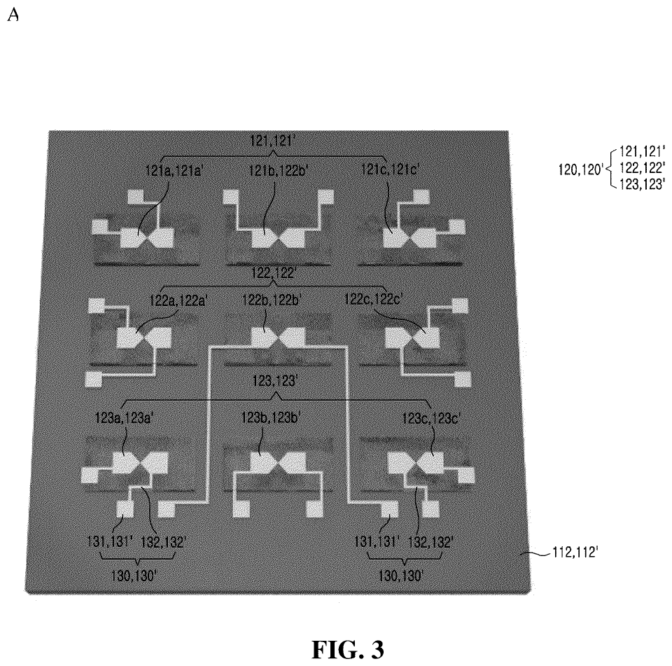

The silicon wafer 112 is formed on the upper surface of the substrate 111, and the sensor portion 120, the plurality of electrode portions 130, and the multi-channel portion 140 are combined. FIG. 3 is a perspective view from one direction actually simulating FIG. 2.

Referring to FIGS. 2 and 3, the sensor portion 120 includes a plurality of sensors 121, 122, 123 arranged in a matrix on the substrate portion 110.

The plurality of sensors 121, 122, 123 generate different electric signals for target materials and transmit them to the plurality of electrode portions 130, respectively.

Here, the different electric signals are different electrical reactive signals.

The plurality of sensors 121, 122, 123 interact with the target materials to generate different electrical reactive signals for detecting the cause materials of a degenerative neurological disease and transmit them to the plurality of electrode portions 130, respectively.

Specifically, the plurality of sensors 121, 122, 123 are made of different graphene oxides and generate different electrical reactive signals due to changes in the properties of a graphene oxide (GO) through an interaction with the target materials.

The above-described plurality of sensors 121, 122, 123 are an upper sensor 121, a central sensor 122, and a lower sensor 123, as shown in FIGS. 2 and 3.

The upper sensor 121 includes a first upper sensor 121a, a second upper sensor 121b, and a third upper sensor 121c.

The upper sensor 121 is formed on the upper surface of the substrate portion 110 and arranged in row 1.

The above-described upper sensor 121 includes a first upper sensor 121a, a second upper sensor 121b, and a third upper sensor 121c that are sequentially arranged in row 1.

The first upper sensor 121a is formed on one upper side of the central portion of the silicon wafer 112.

The second upper sensor 121b is formed in the central upper portion at the central portion of the silicon wafer 112 so as to be positioned on the other side of the first upper sensor 121a.

The third upper sensor 121c is formed in the other side upper portion at the central portion of the silicon wafer 112 so as to be positioned on the other side of the second upper sensor 121b.

The central sensor 122 is formed on the upper surface of the substrate portion 110 in row 2 so as to be positioned below the upper sensor 121.

The central sensor 122 includes the first central sensor 122a, the second central sensor 122b, and the third central sensor 122c sequentially arranged in row 2.

The first central sensor 122a is formed in the central portion of one side of the silicon wafer 112 so as to be positioned below the first upper sensor 121a.

The second central sensor 122b is formed at the center at the central portion of the silicon wafer 112 so as to be positioned on the other side of the first central sensor 122a and below the second upper sensor 121b.

The third central sensor 122c is formed at the center at the central portion of the other side of the silicon wafer 112 so as to be positioned on the other side of the second central sensor 122b and below the third upper sensor 121c.

The lower sensor 123 is formed on the upper surface of the substrate portion 110 in row 3 so as to be positioned below the central sensor 122.

The lower sensor 123 described above includes a first lower sensor 123a, a second lower sensor 123b, and a third lower sensor 123c sequentially arranged in row 3.

The first lower sensor 123a is formed in the other side lower side at the central portion of the silicon wafer 112 so as to be positioned below the first central sensor 122a.

The second lower sensor 123b is formed in the central lower side at the central portion of the silicon wafer 112 so as to be positioned below the second central sensor 122b and on the other side of the first lower sensor 123a.

The third lower sensor 123c is formed in the other side lower side at the central portion of the silicon wafer 112 so as to be positioned below the third central sensor 122c and at the other side of the second lower sensor 123b.

The above-described sensor portion 120 is composed of a total of nine sensors.

That is, the sensor portion 120 is composed of first to third upper sensors 121a, 121b, 121c, first to third central sensors 122a, 122b, 122c, and first to third lower sensors 123a, 123b, 123c.

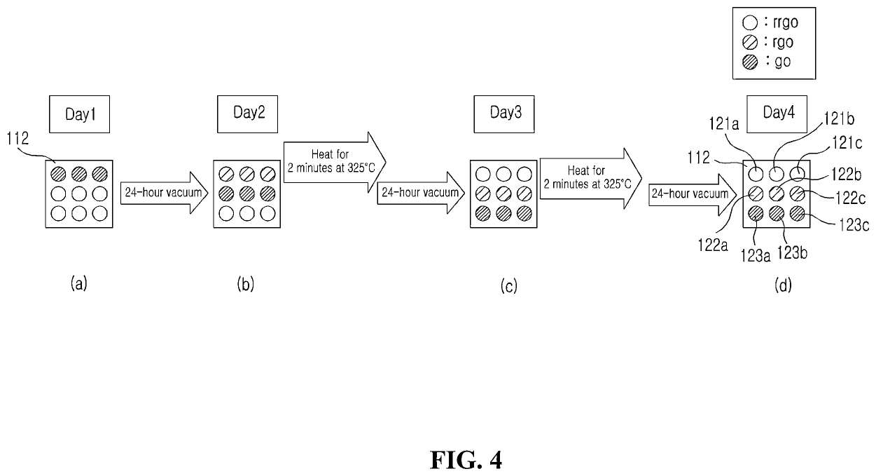

FIG. 4 ((a), (b), (c), and (d)) is schematic views showing a process of forming a sensor portion provided in a multi-sensor according to an embodiment of the disclosure.

In addition, the upper sensor 121 described above is a secondary reduced graphene oxide (rrgo), the central sensor 122 is a primary reduced graphene oxide (rgo), the lower sensor 123 is a graphene oxide (go), and referring to FIG. 3, a process of forming the sensor portion 120 on the upper surface of the silicon wafer 112 is explained.

First, referring to (a) of FIG. 4, on the first day, 1.5 ul of a graphene oxide (go) is sequentially dropped on three positions in row 1 on the upper surface of the silicon wafer 112, and then stabilized in a desiccator for 24 hours.

Next, referring to (b) of FIG. 4, on the second day, the stabilized graphene oxide (go) in the row 1 is baked in an oven at a temperature of 325° C. for 2 minutes to create a primary graphene oxide (rgo), and then the graphene oxide (go) is sequentially dropped again on three positions in row 2 of the upper surface of the silicon wafer 112, and the primary graphene oxide (rgo) in row 1 and the graphene oxide (go) in row 2 are stabilized in a desiccator for 24 hours.

Next, referring to (c) of FIG. 4, on the third day, the stabilized row 1 primary graphene oxide (rgo) and the stabilized row 2 primary graphene oxide (go) are baked in an oven at a temperature of 325° C. for 2 minutes to create a row 2 secondary graphene oxide (rrgo) and row 2 primary graphene oxide (rgo), respectively, and then the graphene oxide (go) is sequentially dropped again on three places in row 3 on the upper surface of the silicon wafer 112, and the row 2 secondary graphene oxide (rrgo), row 2 primary graphene oxide (rgo), and row 3 primary graphene oxide (go) are stabilized in a desiccator for 24 hours.

Through the above process, as shown in (d) of FIG. 4, the first to third upper sensors 121a, 121b, 121c, first to third central sensors 122a, 122b, 122c, and first to third lower sensors 123a, 123b, 123c described above are formed.

Referring to FIGS. 2, 3, and 4 ((d)), the first to third upper sensors 121a, 121b, 121c, the first to third central sensors 122a, 122b, 122c, and the first to third lower sensors 123a, 123b, 123c are arranged in a 3×3 matrix.

In addition, although the disclosure describes a total of nine sensors, this may be changed and applied at any time according to a user's needs or purposes, and as the number of sensors increases, the accuracy of diagnosing Alzheimer's disease and Parkinson's disease can be improved. As described above, the upper sensor 121, central sensor 122, and lower sensor 123 are a secondary reduced graphene oxide (rrgo: reduced reduced graphene oxide), primary reduced graphene oxide (rgo), and graphene oxide (go: graphene oxide) generated from the same graphene oxide under different conditions, and therefore, when they interact with the target materials, they generate different electrical reactive signals due to changes in the physical properties of the graphene oxide.

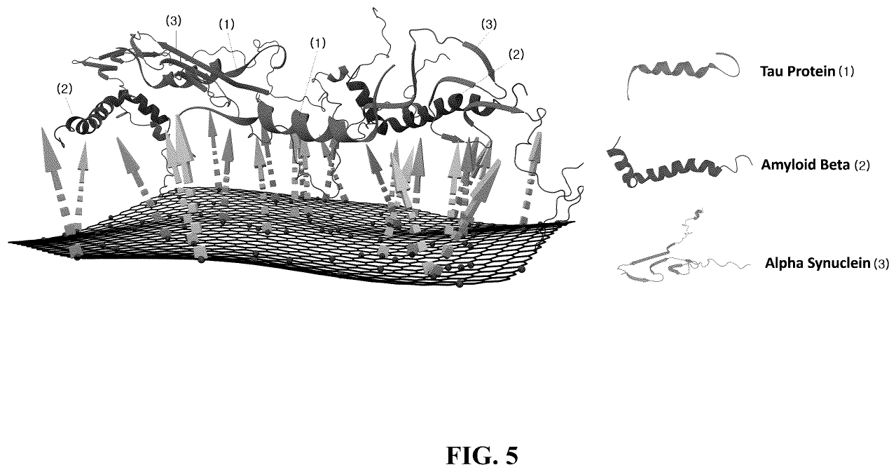

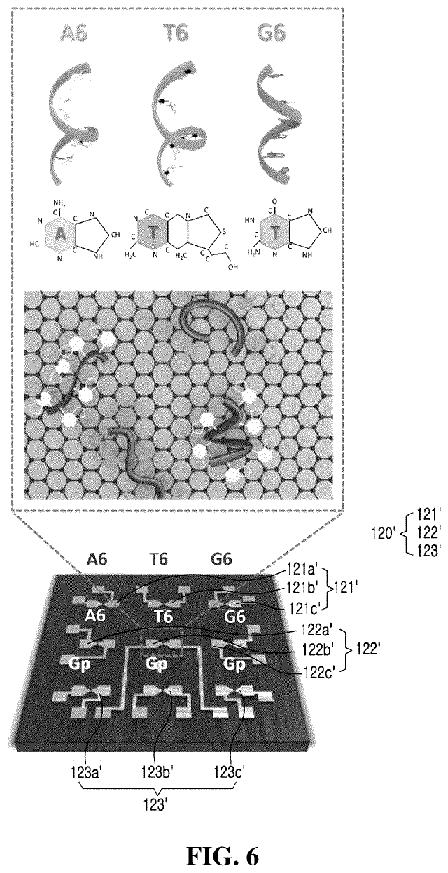

FIG. 5 is a conceptual view showing an interaction between a target material and a sensor portion provided in a multi-sensor according to a first embodiment of the disclosure.

The above-described target material, which is liquid protein separated from blood, includes alpha synuclein (AS), beta amyloid (AB), and tau protein (TP), as shown in FIG. 5.

Specifically, alpha synuclein, beta amyloid, and tau protein are a type of brain protein, so they may be expressed in a shape similar to DNA, as shown in FIG. 5.

Referring to FIGS. 2 and 3, the plurality of electrode portions 130 are arranged radially from the center of the sensor portion 120 to the substrate portion 110 and are electrically connected to the plurality of sensors 121, 122, 123.

The above-described plurality of electrode portions 130 include a plurality of electrodes 131 and a plurality of connection electrodes 132.

The plurality of electrodes 131 are arranged on the edge of the substrate portion 110.

Specifically, four electrodes 131 are formed on the upper edge of the silicon wafer 112 (hereinafter, referred to as first to fourth electrodes sequentially from the left side of FIGS. 2 and 3), and six electrodes 131 are formed on the lower edge of the silicon wafer 112 (hereinafter, referred to as fifth to tenth electrodes sequentially from the left side of FIGS. 2 and 3).

In addition, four electrodes 131 are formed on one edge of the silicon wafer 112 (hereinafter, referred to as eleventh to fourteenth electrodes sequentially from the top side of FIGS. 2 and 3), and four electrodes 131 are formed on the other edge of the silicon wafer 112 (hereinafter, referred to as fifteenth to eighteenth electrodes sequentially from the top side of FIGS. 2 and 3).

The plurality of connection electrodes 132 are configured (hereinafter, referred to as first to eighteenth connection electrodes in a clockwise direction from the point where they are connected to the first upper sensor 121a of FIG. 2) so as to be electrically connected in pairs to the first to third upper sensors 121a, 121b, 121c, the first to third central sensors 122a, 122b, 122c, and the first to third lower sensors 123a, 123b, 123c.

Specifically, the first and second connection electrodes 132 electrically connect the eleventh and first electrodes 131 to the first upper sensor 121a, respectively.

In addition, the third and fourth connection electrodes 132 electrically connect the second and third electrodes 131 to the second upper sensor 121b, respectively.

In addition, the fifth and sixth connection electrodes 132 electrically connect the fourth and fifteenth electrodes 131 to the third upper sensor 121c, respectively.

In addition, the seventeenth and eighteenth connection electrodes 132 electrically connect the thirteenth and twelfth electrodes 131 to the first central sensor 122a, respectively.

In addition, the eleventh and 14th connection electrodes 132 electrically connect the ninth and sixth electrodes 131 to the second central sensor 122b, respectively.

In addition, the seventh and eighth connection electrodes 132 electrically connect the sixteenth and seventeenth electrodes 131 to the third central sensor 122c, respectively.

In addition, the fifteenth and sixteenth connection electrodes 132 electrically connect the fifth and fourteenth electrodes 131 to the first lower sensor 123a, respectively.

In addition, the twelfth and thirteenth connection electrodes 132 electrically connect the eighth and seventh electrodes 131 to the second lower sensor 123b, respectively.

In addition, the ninth and tenth connection electrodes 132 electrically connect the eighteenth and tenth electrodes 131 to the third lower sensor 123c, respectively.

Referring to FIGS. 1 and 2, the multi-channel portion 140 has a plurality of body holes 142, 143, 144 which are positioned above a plurality of sensors 121, 122, 123 and into which target materials are introduced.

Referring to FIG. 1, the multi-channel portion 140 includes a body 141, an upper body hole 142, a central body hole 143, and a lower body hole 144.

The body 141 is coupled to the upper central portion of the substrate portion 110 so as to be positioned above the sensor portion 120.

Specifically, the body 141 may have a hexahedral shape having a PDMS structure.

The upper body hole 142 is formed to penetrate the body 141 in the vertical direction.

The above-described upper body hole 142 includes a first upper body hole 142a, a second upper body hole 142b, and a third upper body hole 142c.

The first upper body hole 142a is formed to penetrate the body 141 in the vertical direction so as to be positioned above the first upper sensor 121a.

The second upper body hole 142b is formed to penetrate the body 141 in the vertical direction so as to be positioned above the second upper sensor 121b.

The third upper body hole 142c is formed to penetrate the body 141 in the vertical direction so as to be positioned above the third upper sensor 121c.

The central body hole 143 is formed to penetrate the body 141 in the vertical direction.

The above-described central body hole 143 includes a first central body hole 143a, a second central body hole 143b, and a third central body hole 143c.

The first central body hole 143a is formed to penetrate the body 141 in the vertical direction so as to be positioned above the first central sensor 121a.

The second central body hole 143b is formed to penetrate the body 141 in the vertical direction so as to be positioned above the second central sensor 122b.

The third central body hole 143c is formed to penetrate the body 141 in the vertical direction so as to be positioned above the third central sensor 122c.

The lower body hole 144 is formed to penetrate the body 141 in the vertical direction.

The above-described lower body hole 144 includes a first lower body hole 144a, a second lower body hole 144b, and a third lower body hole 144c.

The first lower body hole 144a is formed to penetrate the body 141 in the vertical direction so as to be positioned above the first lower sensor 123a.

The second lower body hole 144b is formed to penetrate the body 141 in the vertical direction so as to be positioned above the second lower sensor 123b.

The third lower body hole 144c is formed to penetrate the body 141 in the vertical direction so as to be positioned above the third lower sensor 123c.

### 2. Multi-Sensor (100′)—Second Embodiment

Hereinafter, a multi-sensor according to a second embodiment of the disclosure will be described with reference to FIGS. 1 to 3 and FIG. 6.

Since the second embodiment has substantially the same components as the first embodiment, a detailed description thereof will be brief.

However, since the second embodiment differs from the first embodiment in terms of the materials that make up the plurality of sensors, a detailed description thereof will be given.

Referring to FIGS. 1 and 2, a multi-sensor 100′ according to the second embodiment of the disclosure includes a substrate portion 110′, a sensor portion 120′, an electrode portion 130′, and a multi-channel portion 140′, and may also be implemented as a portable device.

Referring to FIG. 1, the substrate portion 110′ includes a substrate 111′ and a silicon wafer 112′.

Referring to FIGS. 2 and 3, the sensor portion 120′ includes a plurality of sensors 121′, 122′, 123′ arranged in a matrix on the substrate portion 110′.

Specifically, the sensor portion 120′ directly contacts an analysis target gas to generate nine different electrochemical signals.

The above-described sensor portion 120′ may simultaneously detect the characteristics of various types of analysis target gases by arranging a plurality of sensors 121′, 122′, 123′ in an array form.

The plurality of gas sensors 121′, 122′, 123′ show high sensitivity to a specific gas or gas group and may perform stably even under various environmental conditions.

In addition, the sensor portion 120′ may be connected to a measurement circuit that may precisely measure an electrochemical signal that varies depending on the composition ratio and type of the analysis target gas.

The plurality of sensors 121′, 122′, 123′ generate different electric signals for target materials and transmit them to the plurality of electrode portions 130′.

Here, the different electric signals are different electrochemical signals.

Specifically, the plurality of sensors 121′, 122′, 123′ perform a role in which the electrical characteristics change due to gas adsorption, and the type of gas sensor is not particularly limited as long as it performs the above function.

In addition, the plurality of sensors 121′, 122′, 123′ preferably include one or more two-dimensional materials selected from the group including ssDNA-bound graphene, single graphene, graphene oxide, MXene, and metal oxide, but are not limited thereto, and other possible plurality of sensors 121′, 122′, 123′ may include at least one of a metal oxide, nanotube, nanowire, etc.

Here, the ssDNA-bound graphene includes a graphene functionalized with one type of ssDNA and a graphene functionalized with two types of ssDNA.

Specifically, a plurality of sensors 121′, 122′, 123′ generate different electrochemical signals for diagnosing one of the diseases of the liver, kidney, gastric ulcer, duodenal ulcer, odor-related disease, lung cancer, and pancreatitis, and transmit them to a plurality of electrode portions 130′.

Here, the target materials are an analysis target gas, which is an animal's exhaled gas (more specifically, the target material are a human's exhaled gas), and include ammonia (NH3), hydrogen sulfide (H₂S), and carbon monoxide NO.

The composition of the analysis target gas may include the composition of a disease biomarker gas.

Here, the disease biomarker gas refers to a specific gas that is an indicator that can determine the occurrence, progression, or condition of a disease. Such a disease biomarker gas is detected only in the exhaled breath of patients with a specific disease, or its composition ratio shows a difference between normal people and patients.

For example, in some cancers, the composition ratio of a specific compound is observed to increase, and this may be used to determine the early diagnosis or progression of the cancer.

In addition, it is known that the composition ratio of acetone increases in the exhaled breath of diabetic patients, and this may be used to check the management and progression of diabetes.

In addition, specific biomarker gases are found in liver disease, kidney disease, lung disease, etc., and this may assist in the diagnosis and treatment of the corresponding diseases.

The types of disease biomarker gases that perform the above functions are not particularly limited.

In addition to the above, possible disease biomarker gases may include methanol, acetaldehyde, acetic acid, ethanol, methyl ethyl ketone, propionic acid, toluene, etc. In addition, each disease biomarker gas may be directly related to the incidence of a specific disease, and the composition ratio and type of these gases may be used to predict the patient's health status and the possibility of disease occurrence.

In the disclosure, nine sensors (three upper sensors, three central sensors, and three lower sensors) are described as detecting nine different electrochemical signals, but considering performance and cost, only six of the nine sensors may be used.

TABLE 1

| Material | |
|---|---|
| name | Materials |
| Gp | Graphene |
| A6 | Graphene + Adenine 6mer |
| G6 | Graphene + Guanine 6mer |
| T6 | Graphene + Thymine 6mer |
| AT | Graphene + Adenine 6mer + Thymine |
| | 6mer |
| AG | Graphene + Adenine 6mer + Guanine |
| | 6mer |
| TG | Graphene + Guanine 6mer + Thymine |
| | 6mer |

Specifically, deoxyribose (sugar) of DNA has an H group, which may greatly increase the reactivity with gas molecules, water molecules, and electrode materials.

Accordingly, ssDNA, which is 6mer (composed of 6 nucleobases), was functionalized for graphene array sensing.

As the sequence length of ssDNA increases, the production cost increases, but 6mer already shows sufficient performance.

FIG. 6 is a view showing a graphene functionalized with one type of ssDNA, a graphene functionalized with two types of ssDNA, and a single graphene forming a multi-sensor and an upper sensor, a central sensor, and a lower sensor provided in the multi-sensor according to a second embodiment of the disclosure.

The above-described plurality of sensors 121′, 122′, 123′ are an upper sensor 121′, a central sensor 122′, and a lower sensor 123′ as shown in FIGS. 2, 3, and 6.

The upper sensor 121′ includes a first upper sensor 121a′, a second upper sensor 121b′, and a third upper sensor 121c′.

The upper sensor 121′ is formed on the upper surface of the substrate portion 110′ and is arranged in row 1.

Specifically, the upper sensor 121′ may be a graphene functionalized with one type of ssDNA.

Referring to FIG. 6, the above-described upper sensor 121′ includes a first upper sensor 121a′, a second upper sensor 121b′, and a third upper sensor 121c′ that are sequentially arranged in row 1.

Referring to FIG. 6, the first upper sensor 121a′ is formed on one upper side of the central portion of the silicon wafer 112, and this first upper sensor 121a′ includes A6 described in [Table 1].

Referring to FIG. 6, the second upper sensor 121b′ is formed on the central upper side of the silicon wafer 112′ so as to be positioned on the other side of the first upper sensor 121a′, and this second upper sensor 121b′ includes T6 described in [Table 1].

Referring to FIG. 6, the third upper sensor 121c′ is formed on the other side upper side of the central portion of the silicon wafer 112′ so as to be positioned on the other side of the second upper sensor 121b′, and this third upper sensor 121c′ includes G6 described in [Table 1].

The central sensor 122′ is formed on the upper surface of the substrate portion 110′ by being arranged in row 2 so as to be positioned below the upper sensor 121′.

Specifically, the central sensor 122′ may be a graphene functionalized with two types of ssDNA.

Referring to FIG. 6, the central sensor 122′ includes a first central sensor 122a′, a second central sensor 122b′, and a third central sensor 122c′ sequentially arranged in row 2.

Referring to FIG. 6, the first central sensor 122a′ is formed at the center of one side of the silicon wafer 112′ so as to be positioned below the first upper sensor 121a′, and this first central sensor 122a′ includes AT.

Referring to FIG. 6, the second central sensor 122b′ is formed at the center of the central portion of the silicon wafer 112′ so as to be positioned on the other side of the first central sensor 122a′ and below the second upper sensor 121b′, and this second central sensor 122b′ includes AG.

Referring to FIG. 6, the third central sensor 122c′ is formed at the other side center of the central portion of the silicon wafer 112′ so as to be positioned on the other side of the second central sensor 122b′ and below the third upper sensor 121c′, and this third central sensor 122c′ includes TG.

The lower sensor 123′ is formed on the upper surface of the substrate portion 110′ by being arranged in row 3 so as to be positioned below the central sensor 122′.

Specifically, the lower sensor 123′ may be a single graphene.

Referring to FIG. 6, the above-described lower sensor 123′ includes a first lower sensor 123a′, a second lower sensor 123b′, and a third lower sensor 123c′ sequentially arranged in row 3.

Referring to FIG. 6, the first lower sensor 123a′ is formed at the other side lower side of the central portion of the silicon wafer 112′ so as to be positioned below the first central sensor 122a′, and this first lower sensor 123a′ includes Gp described in [Table 1].

Referring to FIG. 6, the second lower sensor 123b′ is formed at the lower side of the central portion of the silicon wafer 112′ so as to be positioned below the second central sensor 122b′ and the other side of the first lower sensor 123a′, and this second lower sensor 123′ includes Gp described in [Table 1].

Referring to FIG. 6, the third lower sensor 123c′ is formed on the other side lower side of the central portion of the silicon wafer 112′ so as to be positioned below the third central sensor 122c′ and on the other side of the second lower sensor 123b′, and this third lower sensor 123c′ includes Gp described in [Table 1].

The above-described sensor portion 120′ may be composed of a total of 9 sensors.

That is, the sensor portion 120′ is composed of the first to third upper sensors 121a′, 121b′, 121c′, the first to third central sensors 122a′, 122b′, 122c′, and the first to third lower sensors 123a′, 123b′, 123c′.

The electrode portion 130′ is arranged radially from the center of the sensor portion 120′ to the substrate portion 110′ and is electrically connected to a plurality of sensors 121′, 122′, 123′, and is composed of a plurality of electrode portions 130′.

The above-described plurality of electrode portions 130′ includes a plurality of electrodes 131′ and a plurality of connection electrodes 132′

Referring to FIG. 1, the multi-channel portion 140′ has a plurality of body holes 142′, 143′, 144′ positioned above a plurality of sensors 121′, 122′, 123′ into which target materials are introduced.

Referring to FIG. 1, the multi-channel portion 140′ includes a body 141′, an upper body hole 142′, a central body hole 143′, and a lower body hole 144′.

The upper body hole 142′ is formed to penetrate the body 141′ in the vertical direction.

The upper body hole 142′ includes a first upper body hole 142a′, a second upper body hole 142b′, and a third upper body hole 142c′.

The first upper body hole 142a′ is formed to penetrate the body 141′ in the vertical direction so as to be positioned above the first upper sensor 121a′.

The second upper body hole 142b′ is formed to penetrate the body 141′ in the vertical direction so as to be positioned above the second upper sensor 121b′.

The third upper body hole 142c′ is formed to penetrate the body 141′ in the vertical direction so as to be positioned above the third upper sensor 121c′.

The central body hole 143′ is formed to penetrate the body 141′ in the vertical direction. The above-described central body hole 143′ includes a first central body hole 143a′, a second central body hole 143b′, and a third central body hole 143c′.

The first central body hole 143a′ is formed to penetrate the body 141′ in the vertical direction so as to be positioned above the first central sensor 121a′.

The second central body hole 143b′ is formed to penetrate the body 141′ in the vertical direction so as to be positioned above the second central sensor 122b′.

The third central body hole 143c′ is formed to penetrate the body 141′ in the vertical direction so as to be positioned above the third central sensor 122c′.

The lower body hole 144′ is formed to penetrate the body 141′ in the vertical direction.

The lower body hole 144′ described above includes the first lower body hole 144a′, the second lower body hole 144b′, and the third lower body hole 144c′.

The first lower body hole 144a′ is formed to penetrate the body 141′ in the vertical direction so as to be positioned above the first lower sensor 123a′.

The second lower body hole 144b′ is formed to penetrate the body 141′ in the vertical direction so as to be positioned above the second lower sensor 123b′.

The third lower body hole 144c′ is formed to penetrate the body 141′ in the vertical direction so as to be positioned above the third lower sensor 123c′.

### 3. Multi-Sensor-Using Early Diagnosis System 400—First Embodiment

Hereinafter, an early diagnosis system using a multi-sensor according to a first embodiment of the disclosure will be described with reference to FIGS. 1 to 5 and FIGS. 7 to FIGS. 11A and 11B.

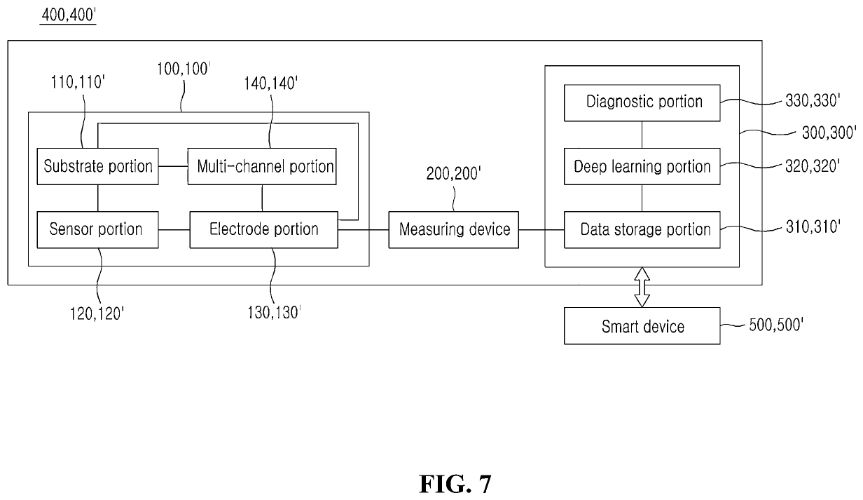

FIG. 7 is a block view showing an early diagnosis system using a multi-sensor according to first and second embodiments of the disclosure.

Referring to FIG. 7, an early diagnosis system 400 using a multi-sensor according to the first embodiment of the disclosure includes a multi-sensor 100, a measuring device 200, and a diagnostic device 300.

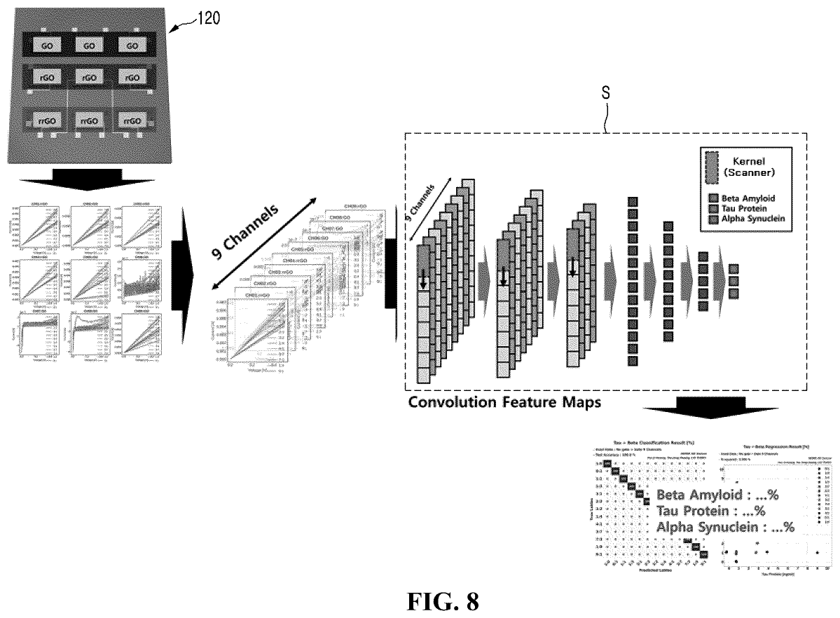

FIG. 8 is a schematic view showing a process of an early diagnosis system using a multi-sensor according to a first embodiment of the disclosure deriving the presence and mixing ratio of a causative material of a degenerative neurological disease through deep learning.

The multi-sensor 100 interacts with target materials flowing into the upper body holes 142a, 142b, 142c, the central body holes 143a, 143b, 143c, and the lower body holes 144a, 144b, 144c, which are the nine channels, to generate different reactive signals, and a schematic view of the contents thereof is illustrated in the upper left of FIG. 8.

In addition, for a specific description of the above-described multi-sensor 100, refer to the above.

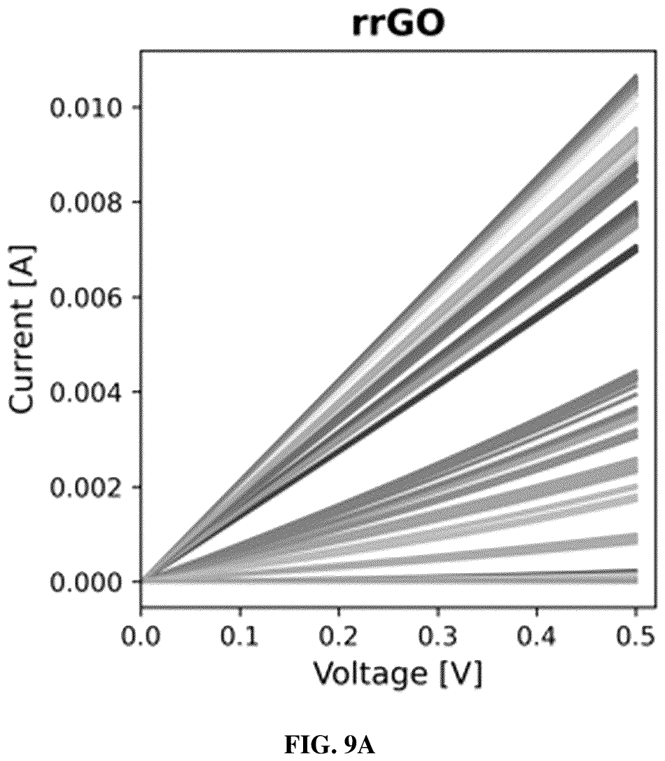

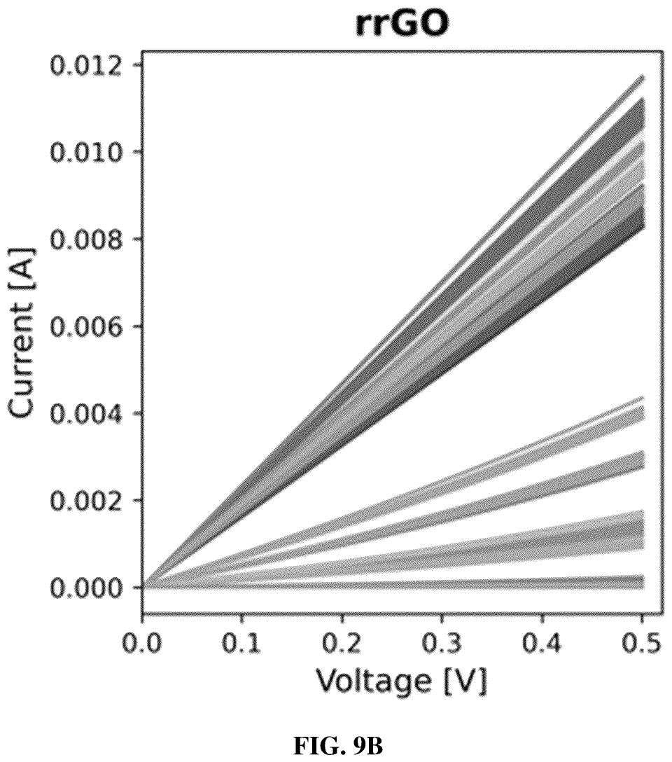

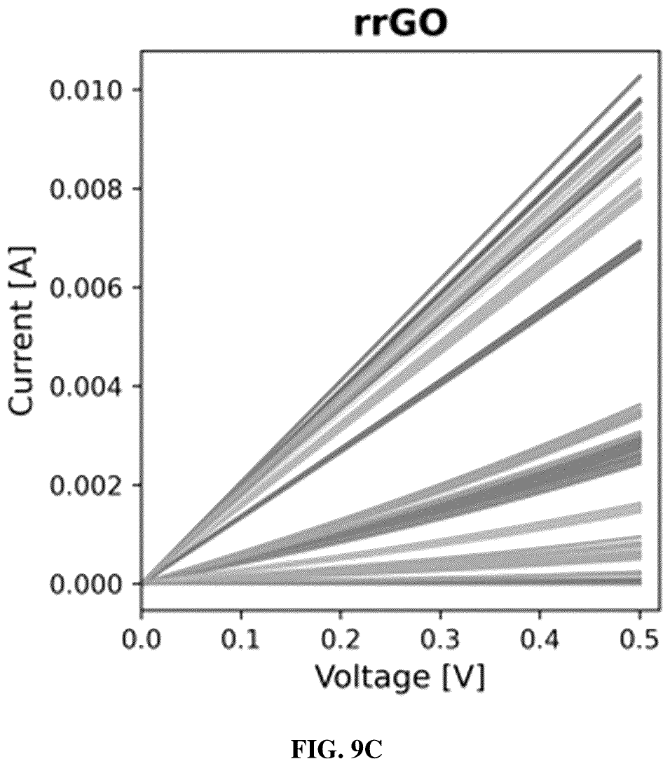

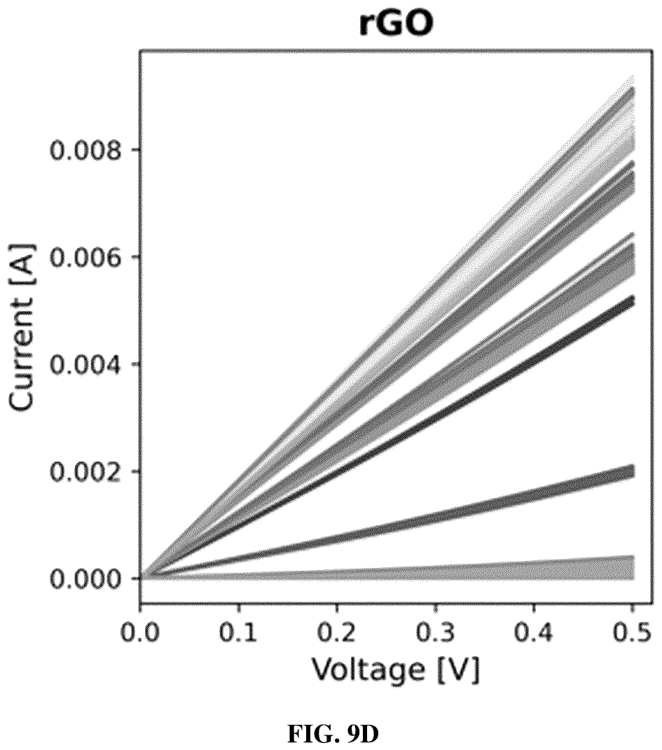

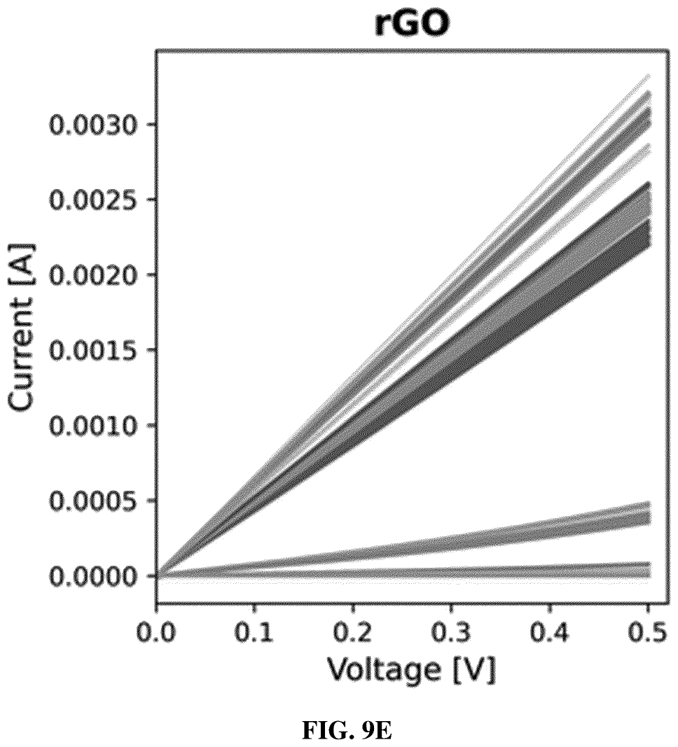

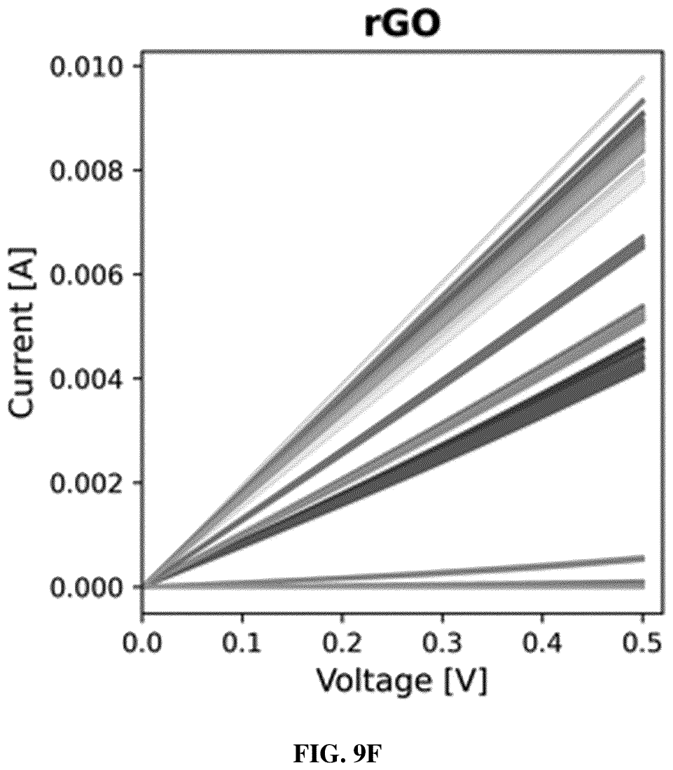

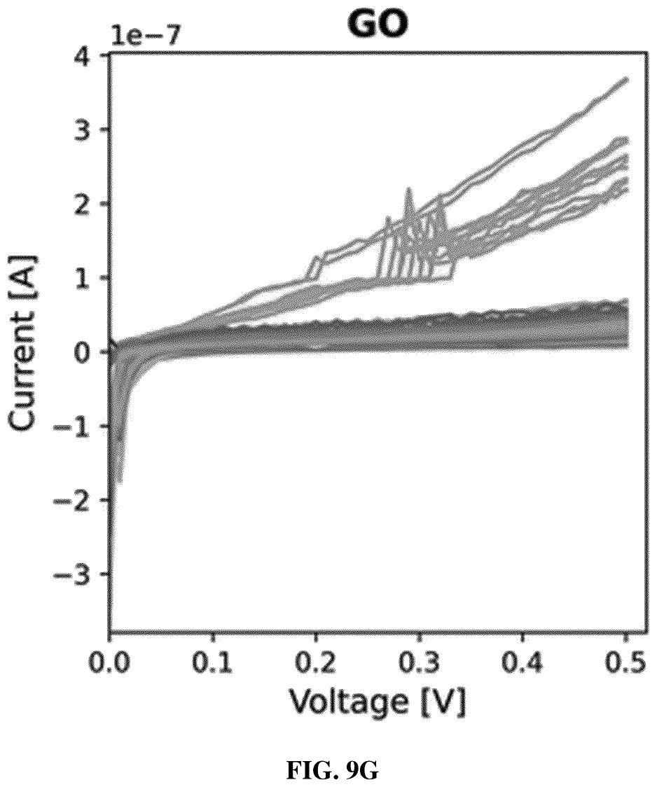

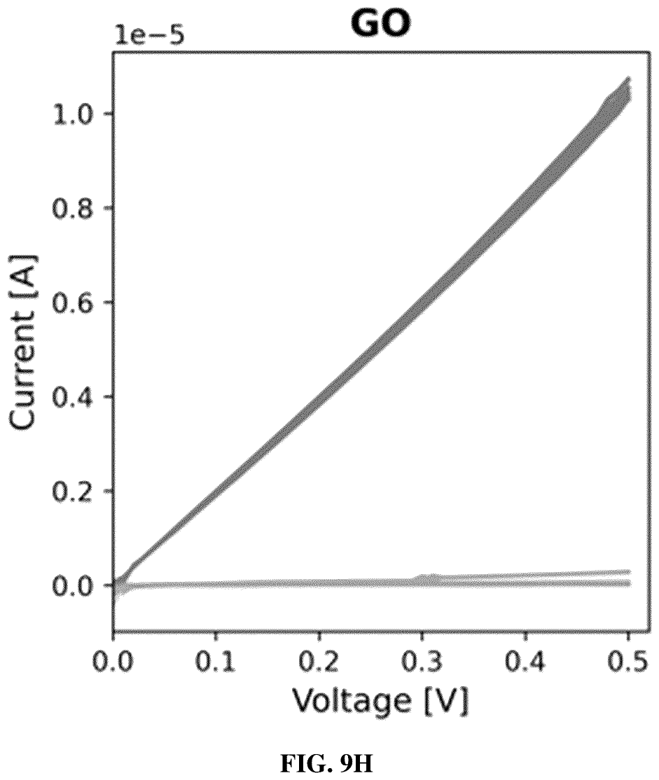

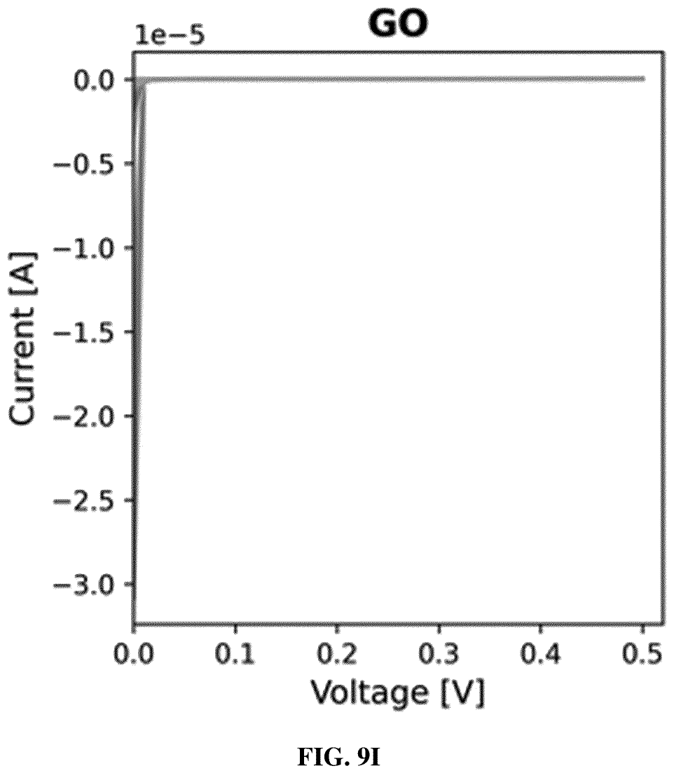

FIGS. 9A, 9B, 9C, 9D, 9E, 9F, 9G, 9H, and 9I are each a graph showing currents according to voltages for nine channels obtained by measuring different reactive signals by a measuring device equipped in an early diagnosis system using a multi-sensor according to first embodiment of the disclosure.

The measuring device 200 is electrically connected to a plurality of electrode portions 130 and measures different electric signals.

Specifically, the measuring device 200 is electrically connected to a plurality of electrode portions 130 and measures different electrical reactive signals, and the measured results are shown in the left side of the center of FIG. 8 and FIGS. 9A, 9B, 9C, 9D, 9E, 9F, 9G, 9H, and 9I.

Specifically, the graphs shown in FIGS. 9A, 9B, 9C, 9D, 9E, 9F, 9G, 9H, and 9I are arranged in the same manner as the plurality of sensors 121, 122, 123 shown in FIGS. 2 and 3 of the first embodiment are arranged in a matrix.

The graphs shown in FIGS. 9A, 9B, and 9C represent currents according to voltages detected by the upper sensor 121 of FIG. 4 made of a secondary reduced graphene oxide (rrgo: reduced reduced graphene oxide), the graphs shown in FIGS. 9D, 9E, and 9F represent currents according to voltages detected by the central sensor 122 of FIG. 4 made of a primary reduced graphene oxide (rgo), and the graphs shown in FIGS. 9G, 9H, and 9I represent currents according to voltages detected by the lower sensor 123 of FIG. 4 made of a graphene oxide (go).

Referring to FIGS. 9A, 9B, 9C, 9D, 9E, 9F, 9G, 9H, and 9I, as the number of reductions in graphene oxide increases, a stable voltage-current graph shape is shown, and the current value is large.

In addition, as shown in FIGS. 9A, 9B, 9C, 9D, 9E, 9F, 9G, 9H, and 9I, graphs for nine different electrical reactive signals may be obtained simultaneously according to the arrangement of three types of sensors, and the nine different electrical reactive signals obtained are input to the deep learning model of FIG. 8.

The diagnostic device 300 inputs different electric signals transmitted from the measuring device 200 into the deep learning model and then diagnoses a disease through regression analysis.

In particular, the diagnostic device 300 improves the diagnostic speed by only using an encoding scheme that increases and then decreases the number of channels in which different electric signals, which are one-dimensional signals, are each grouped.

Here, the different electric signals are different electrical reactive signals.

In addition, the disease includes Alzheimer's disease and Parkinson's disease.

Specifically, the diagnostic device 300 inputs different electrical reactive signals transmitted from the measuring device 200 into a deep learning model and then determines the presence and mixing state of alpha synuclein, beta amyloid, and tau protein, which are the cause materials of degenerative neurological diseases, through regression analysis, thereby simultaneously diagnosing Alzheimer's disease and Parkinson's disease in a label-free manner.

For this, the diagnostic device 300 includes a data storage portion 310, a deep learning portion 320, and a diagnostic portion 330.

The data storage portion 310 stores different electrical reactive signals transmitted from the measuring device 200, and a schematic view thereof is illustrated in the central portion of FIG. 8 (related to 9 channels).

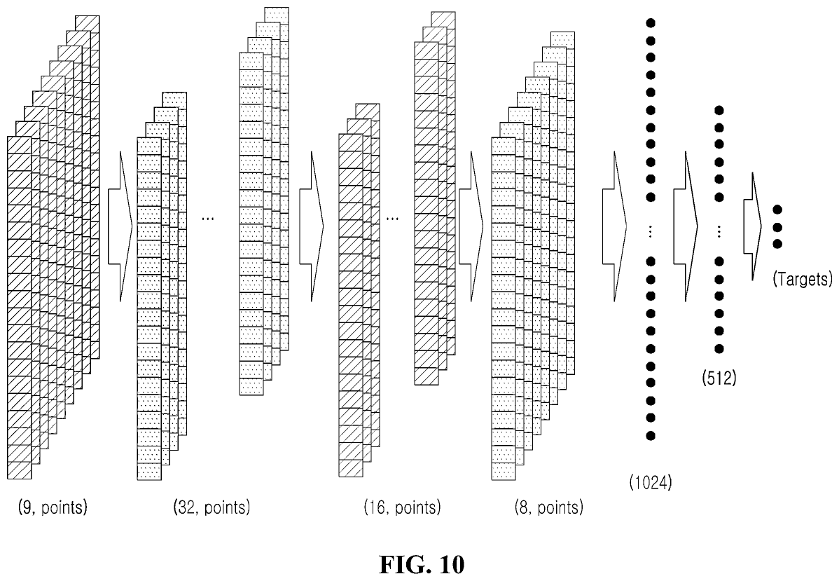

FIG. 10 is a schematic view showing that a deep learning portion equipped in an early diagnosis system using a multi-sensor according to first and second embodiments of the disclosure calculates and performs deep learning on a one-dimensional signal in an encoding manner.

Deep learning is a machine learning technique based on an artificial neural network, and may mean a technology that learns data by imitating the connection structure of the human brain.

The above-described deep learning has been rapidly developing since the 2010s and has been utilized in various fields such as image recognition, voice recognition, natural language processing, computer vision, and recommendation systems.

The core features of deep learning are complex pattern learning through a multi-layer structure, data processing using nonlinear transformation, and accurate learning using a large amount of data.

The deep learning portion 320 inputs different electrical reactive signals transmitted from the data storage portion 310 into a pre-learned deep learning model and performs regression analysis on the output result value, and a schematic view of this is illustrated on the right side of the center of FIG. 8.

Referring to FIG. 10 in more detail, the deep learning portion 320 improves the diagnosis speed by only using an encoding method that increases and then decreases the number of channels in which different electric signals (=signals related to current changes) that are one-dimensional signals (1D signals) are each grouped.

Specifically, referring to (9, point) illustrated in FIG. 10, the deep learning portion 320 measures, n times for a preset time (5 to 6 seconds), nine different electrical reactive signals generated as each target material passing through the first upper body hole 142a, the second upper body hole 142b, the third upper body hole 142c, the first central body hole 143a, the second central body hole 143b, the third central body hole 143c, the first lower body hole 144a, the second lower body hole 144b, and the third lower body hole 144c interacts with the first upper sensor 121a, the second upper sensor 121b, the third upper sensor 121c, the first central sensor 122a, the second central sensor 122b, the third central sensor 122c, the first lower sensor 123a, the second lower sensor 123b, and the third lower sensor 123c.

Here, different electrical reactive signals are depicted as channels, and points are the number of 51 measurements of 9 different electrical reactive signals according to preset voltage intervals 0 to 0.5 V.

Next, referring to (32, point) illustrated in FIG. 10, the deep learning portion 320 increases the number of channels from 9 to 32.

Thereafter, the deep learning portion 320 determines different electrical reactive signals by deep learning by taking an encoding scheme that reduces the number of channels from (32, point) illustrated in FIG. 10 ((32, point)=>(16, point)=>(8, point)).

In order to solve the problem of the conventional technology that takes a long time to calculate in a process of reducing and increasing important characteristics by using an encoding scheme and decoder scheme of reducing the characteristics of a two-dimensional image and leaving only important characteristics (encoder) and then increasing important characteristics again to restore it as a high-resolution two-dimensional image, the above-described deep learning portion 320 may reduce the calculation time and cost by only using an encoding scheme that receives a one-dimensional signal directly and increases the number of channels only at the beginning and then gradually decreases them.

Specifically, the conventional technology performs deep learning by converting the electrical signal data graph for the target response into an image (=2D CNN method) to analyze the two-dimensional image, so the data itself has x, y values and brightness and RGB information, so it is at least in a 3D form.

Accordingly, if the same data is used as an image or a 1D vector, the dimension itself may appear to be the same, but in the case of a two-dimensional image, a blank space is entered as a value of 0, so the data capacity is bound to be heavier, and accordingly, there was a problem that the calculation time increased.

On the other hand, according to the deep learning portion 320 of the disclosure, since the acquired one-dimensional information (=numeric data) is utilized as it is, the one-dimensional information is directly analyzed (=1D CNN) to perform deep learning, thereby improving the computation speed by inputting nine different electrical reactive signals acquired from nine sensors directly into the CNN channel.

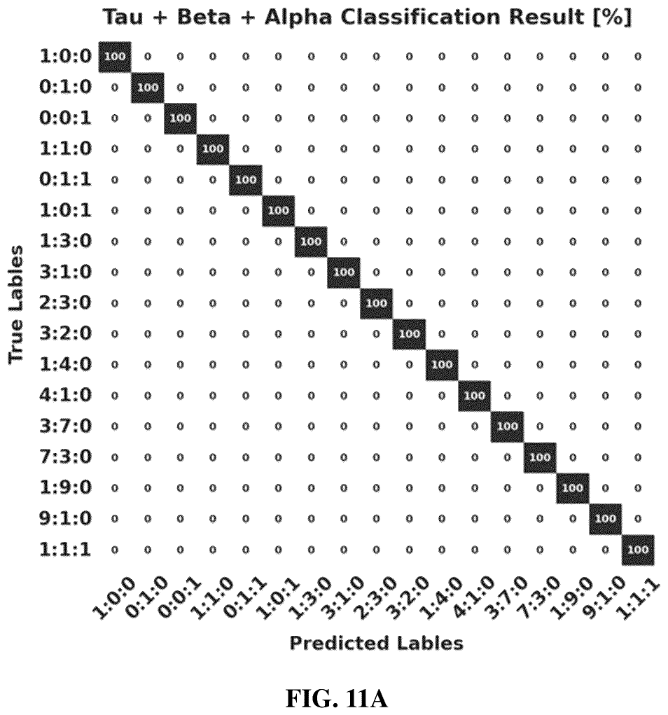

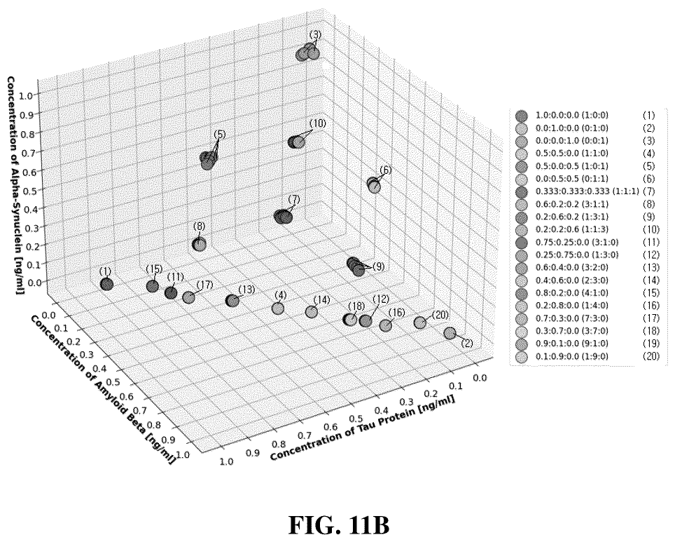

FIGS. 11A and 11B are each a graph showing the accuracy and mixing ratio according to the ratio of alpha synuclein, beta amyloid, and tau protein using a deep learning model in a deep learning portion of an early diagnosis system using a multi-sensor according to a first embodiment of the disclosure.

Referring to FIGS. 11A and 11B, the diagnostic portion 330 simultaneously diagnoses Alzheimer's disease and Parkinson's disease by determining the presence and mixing state of alpha-synuclein, beta-amyloid, and tau protein based on the regression analysis result value transmitted from the deep learning portion 320.

Specifically, the diagnostic portion 330 diagnoses that a patient who provided the target material suffers from Alzheimer's disease when the mixing ratio of alpha-synuclein, beta-amyloid, and tau protein is 3-5:6-14:2-3.

Meanwhile, the diagnostic portion 330 diagnoses that a patient who provided the target material suffers from Parkinson's disease when the mixing ratio of alpha-synuclein, beta-amyloid, and tau protein is 5-13:4-8:1-3.

In addition, the diagnostic device 300 may further include a display unit (not shown) that outputs the results diagnosed by the diagnostic portion 330, and graphs visualized by the display unit are shown in FIG. 8 (current graphs according to voltages of different reactive signals shown on the left side of the center of FIG. 8, and target material mixing ratio graphs shown on the lower right side of FIG. 8) to FIGS. 9A, 9B, 9C, 9D, 9E, 9F, 9G, 9H, and 9I).

Furthermore, the diagnostic device 300 may transmit to a smart device 500 the determination information that determines the presence and mixing state of alpha-synuclein, beta-amyloid, and tau protein diagnosed in the diagnostic portion 330 and the diagnosis information that simultaneously diagnoses Alzheimer's disease and Parkinson's disease, and for this, the diagnostic device 300 may be capable of wireless communication with the smart device 500.

The smart device 500 according to this receives and displays determination information and diagnostic information transmitted from the diagnostic portion 300.

For example, the smart device 500 includes all electronic devices capable of wireless communication, including smartphones, tablet PCs, etc.

### 4. Multi-Sensor-Using Early Diagnosis System 400′

Hereinafter, an early diagnosis system using a multi-sensor according to a second embodiment of the disclosure will be described with reference to FIGS. 1 to 3, FIG. 6, FIGS. 12A, 12B, and 12C to FIGS. 21A, 21B, and 21C.

Referring to FIG. 7, an early diagnosis system 400′ using a multi-sensor according to a second embodiment of the disclosure includes a multi-sensor 100′, a measuring device 200′, and a diagnostic device 300′

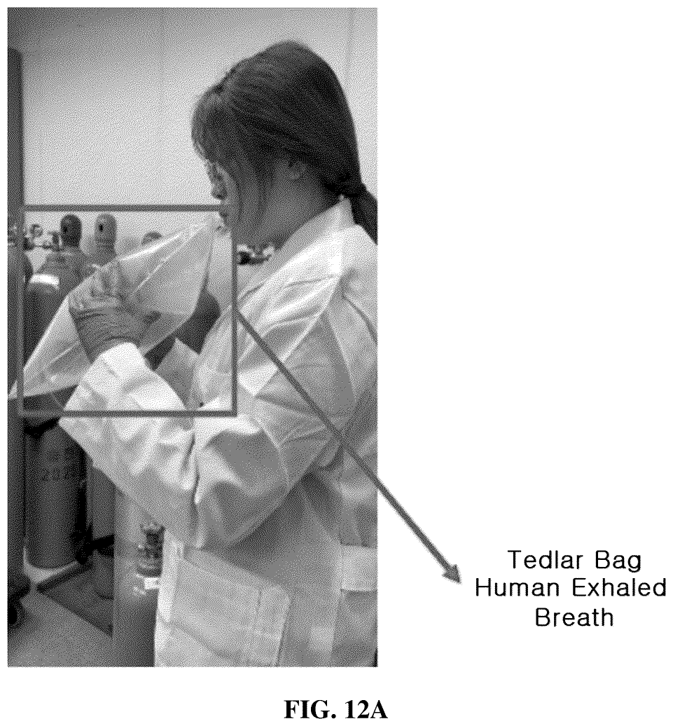

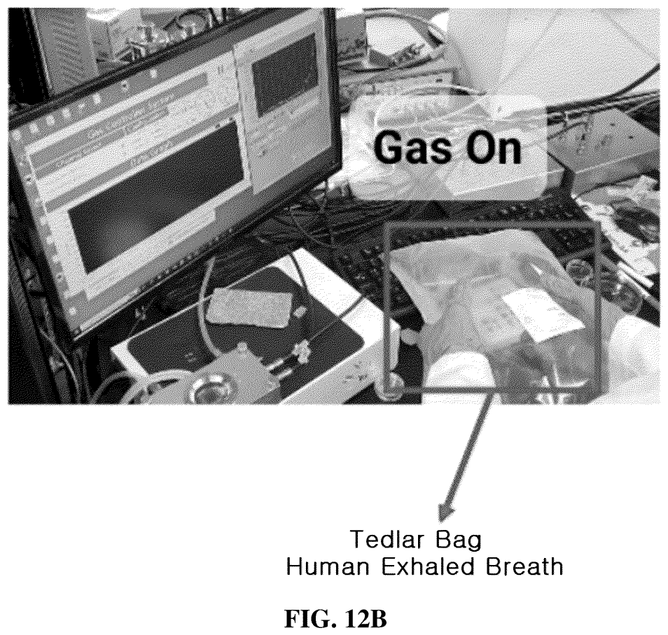

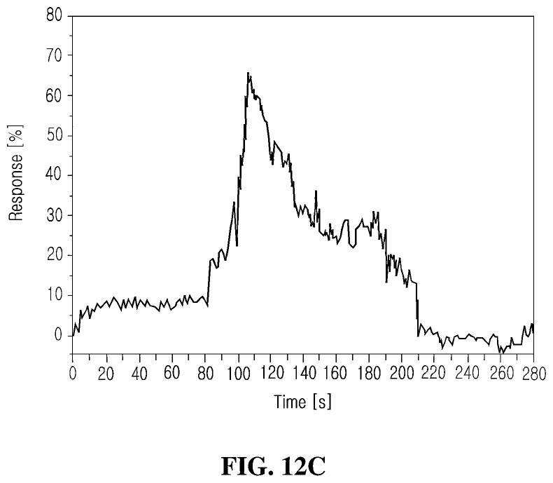

FIG. 12C is a photograph showing the collection of a human exhaled gas using a sampling bag (Tedlar Bag) in an early diagnosis system using a multi-sensor according to a second embodiment of the disclosure. FIG. 12B is a photograph showing the acquisition of different electrochemical signals using a plurality of sensors in an early diagnosis system using a multi-sensor according to a second embodiment of the disclosure.

The disclosure is that an analysis target gas (=exhaled gas) captured by a user through an exhaled gas injection portion (=Tedlar Bag) as shown in FIG. 12A is supplied to the upper portion of a multi-channel portion 140′ equipped in a multi-sensor 100′ as shown in FIG. 12B.

When the analysis target gas is injected into a plurality of sensors using the above-described exhaled gas injection portion, the electrochemical signal change rate is about 70% as a result of an actual experiment, showing high efficiency.

The exhaled gas injection portion (=Tedlar Bag) refers to a bag made of a special polymer material used to store and transport gas or liquid samples.

The exhaled gas injection portion (Tedlar Bag) is chemically stable and is effective in protecting the sample inside from the external environment.

In particular, the exhaled gas injection portion (Tedlar Bag) protects the characteristics of various gas or liquid samples from changing when storing them, and prevents leakage of the sample or cross-contamination with the external environment.

In addition, there is an appropriate connecting device between the exhaled gas inlet (Tedlar Bag) and the multi-channel portion 140′, so that the analysis target gas reaches nine sensors within the multi-channel portion 140′.

The connecting device controls the flow of the analysis target gas, and may change the flow speed or direction of the gas if necessary. This control is necessary to improve the accuracy of the analysis, and keeps the amount of gas reaching each sensor constant.

In addition, the exhaled gas inlet (Tedlar Bag) may have various functions for handling and storing the sample. For example, the exhaled gas inlet (Tedlar Bag) may include an inlet and outlet used when collecting a sample, and a sensor for measuring the composition ratio or pressure of the sample. These functions are used to monitor the characteristics or condition of the sample in real time, and take appropriate actions if necessary.

Accordingly, the analysis target gas discharged from the exhaled gas injection portion passes through the multi-channel portion 140′ and comes into contact with the sensor portion 120 equipped with nine sensors.

Referring to FIG. 1, the multi-sensor 100′ interacts with a target material flowing into the nine channels, the upper body hole 142a′, 142b′, 142c′, the central body hole 143a, 143b, 143c, and the lower body hole 144a, 144b, 144c, to generate different electric signals.

Here, the target material is an analysis target gas, which is an animal's exhaled gas (=human's exhaled gas), and includes ammonia, hydrogen sulfide, and nitrogen monoxide.

In addition, the different electric signals are different electrochemical signals.

In addition, for a specific description of the above-described multi-sensor 100′, refer to the above.

FIG. 12C is a graph for the reactivity over time obtained in response to a multi-sensor in an early diagnosis system using a multi-sensor according to a second embodiment of the disclosure.

The measuring device 200′ is electrically connected to a plurality of electrode portions 130′ to measure different electric signals (=different electrochemical signals), and nine different reactivity signals measured over time through the above process are shown in FIG. 12C.

The diagnostic device 300′ inputs different electric signals (=different electrochemical signals) transmitted from the measuring device 200′ into a deep learning model and then diagnoses a disease through regression analysis.

Referring to FIG. 7, the diagnostic device 300′ includes a data storage portion 310′, a deep learning portion 320′, and a diagnostic portion 330′.

The data storage portion 310′ stores different electrochemical signals transmitted from the measuring device 200′.

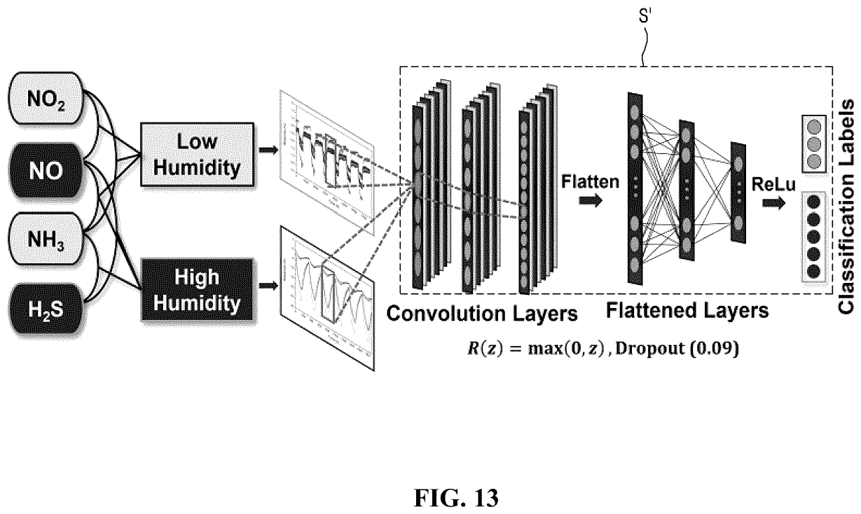

FIG. 13 is a schematic view showing a convolutional neural network (CNN) architecture used for calculating a one-dimensional signal in an encoding manner and deep learning determination in a deep learning portion provided in an early diagnosis system using a multi-sensor according to a second embodiment of the disclosure.

In order to utilize deep learning in the disclosure, the algorithm is pre-learned. In the initial stage, different electrochemical signals may be measured and collected from analysis target gases of various types and compositions, and preprocessed through noise removal and normalization processes. A deep learning model is learned based on the pre-processed data, and this model may accurately learn complex patterns of mixed gases.

In particular, the analysis target gas flowing into a plurality of body holes is measured using a plurality of sensors based on two-dimensional materials, and high discrimination accuracy is guaranteed even in environmental changes through sensors that are not affected by humidity.

Referring to FIG. 13, the deep learning portion 320′ improves the diagnosis speed by only using an encoding scheme that increases and then decreases the number of a plurality of channels in which each of the one-dimensional signals, different electric signals (=different electrochemical signals), is grouped.

Specifically, referring to (9, point) illustrated in FIG. 10, the deep learning portion 320′ measures, n times over a preset time 5 to 6 seconds, nine different electrochemical signals generated as each target material that passes through the first upper body hole 142a, the second upper body hole 142b, the third upper body hole 142c, the first central body hole 143a, the second central body hole 143b, the third central body hole 143c, the first lower body hole 144a, the second lower body hole 144b, and the third lower body hole 144c interacts with the first upper sensor 121a, the second upper sensor 121b, the third upper sensor 121c, the first central sensor 122a, the second central sensor 122b, the third central sensor 122c, the first lower sensor 123a, the second lower sensor 123b, and the third lower sensor 123c.

Here, different electrochemical signals are depicted as channels, and the point is 257, which is the number of times nine different electrochemical signals are measured 200 to 600 times at preset time intervals (5 to 6 seconds).

Next, referring to (32, point) illustrated in FIG. 10, the deep learning portion 320 increases the number of channels from 9 to 32.

Thereafter, the deep learning portion 320′ deep learns different electrical reactive signals by taking an encoding scheme that reduces the number of channels from (32, point) as shown in FIG. 10 ((32, point)=>(16, point)=>(8, point)).

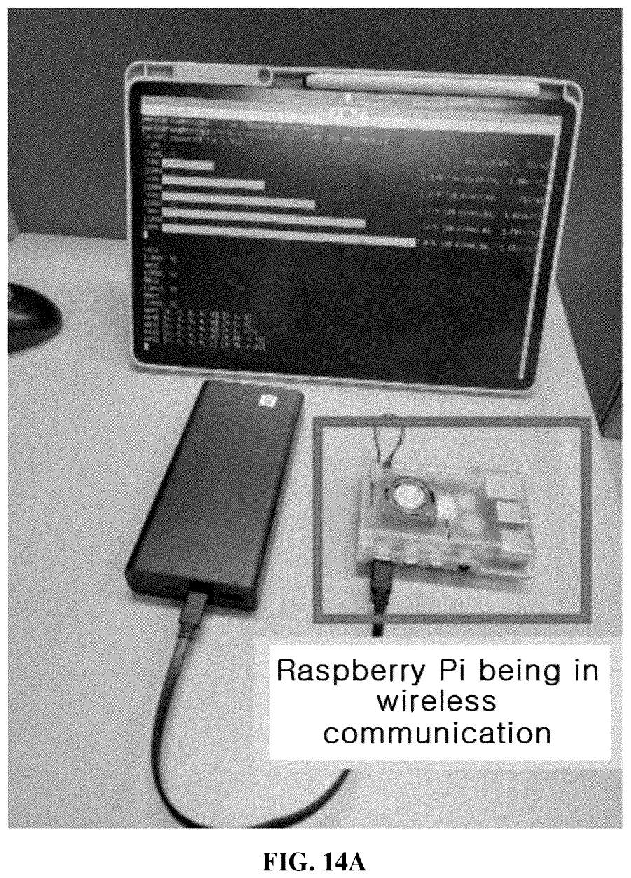

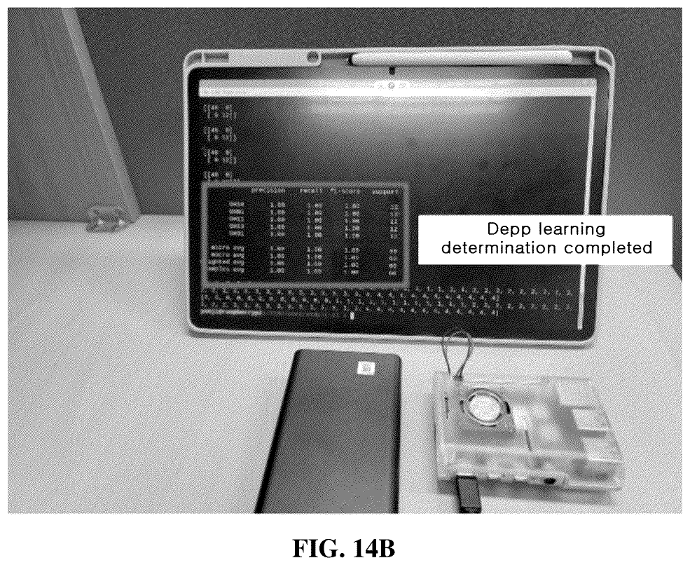

FIG. 14A is a photograph showing wireless communication with a tablet PC, which is a smart device, using a Raspberry Pi in an early diagnosis system using a multi-sensor according to a second embodiment of the disclosure. FIG. 14B is a photograph showing monitoring of a deep learning determination process of an analysis target gas in an early diagnosis system using a multi-sensor according to a second embodiment of the disclosure.

The deep learning portion 320′ may communicate wirelessly with a tablet PC, which is a smart device 500′, using a Raspberry Pi as shown in FIG. 14A.

In addition, the smart device 500′ may monitor the deep learning determination process of the analysis target gas as shown in FIG. 14B.

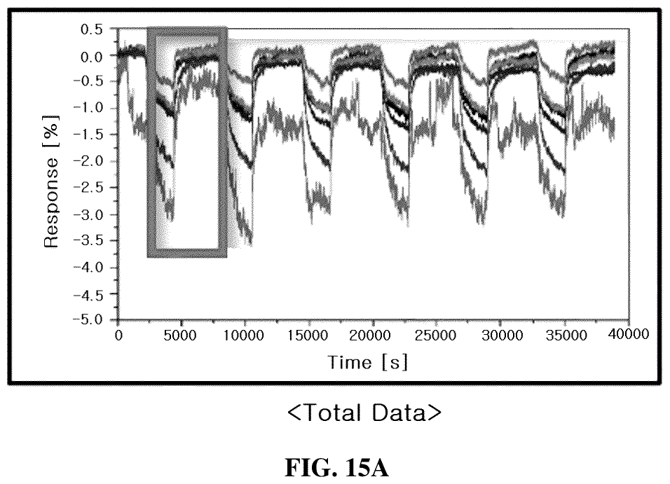

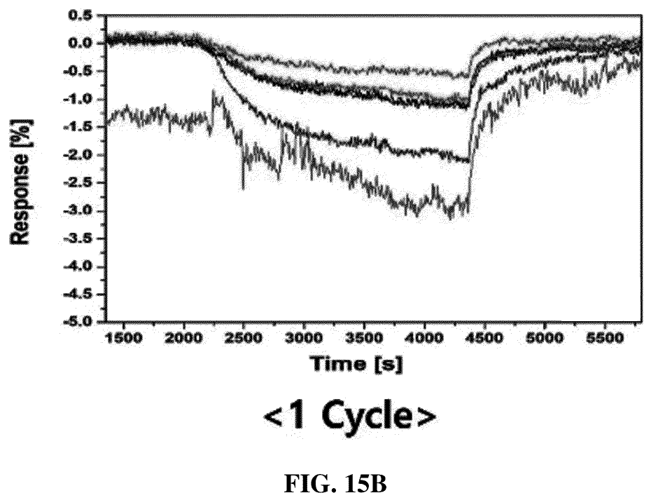

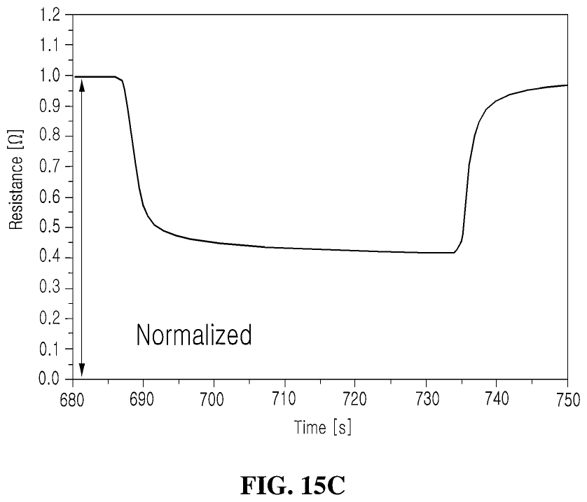

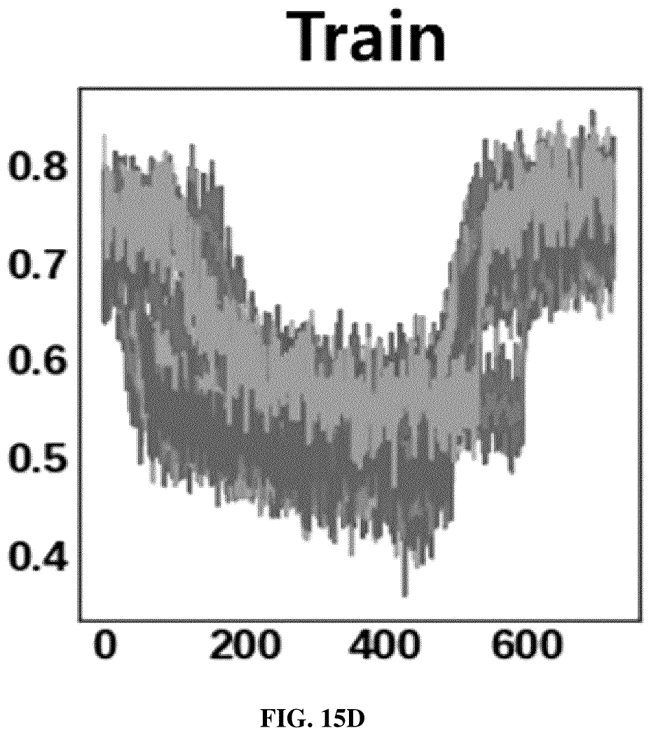

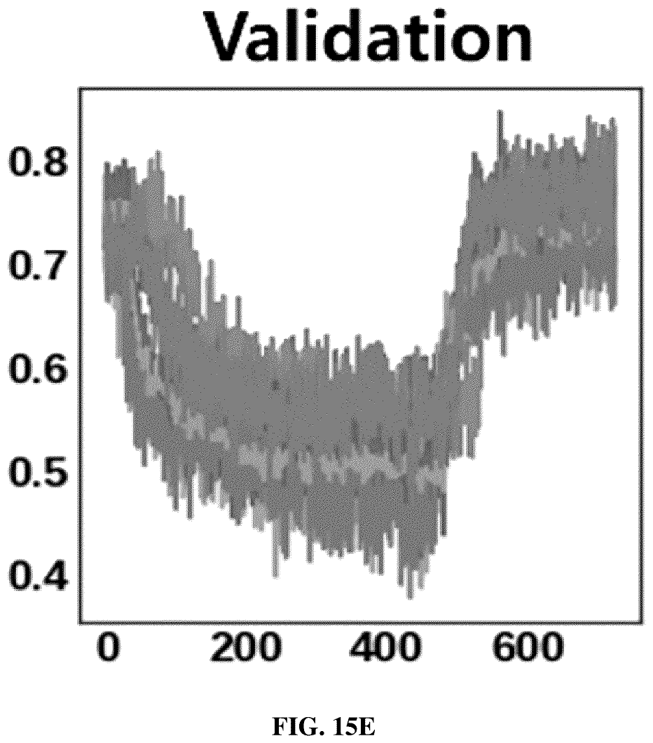

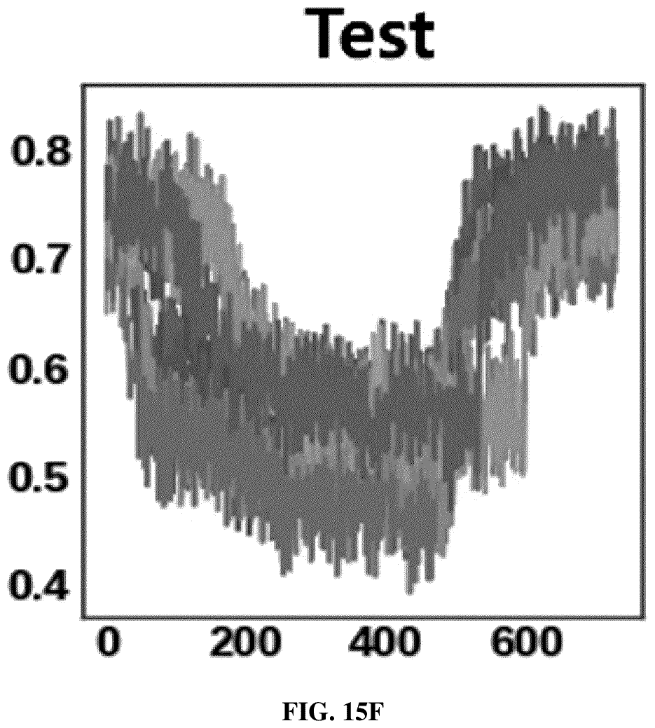

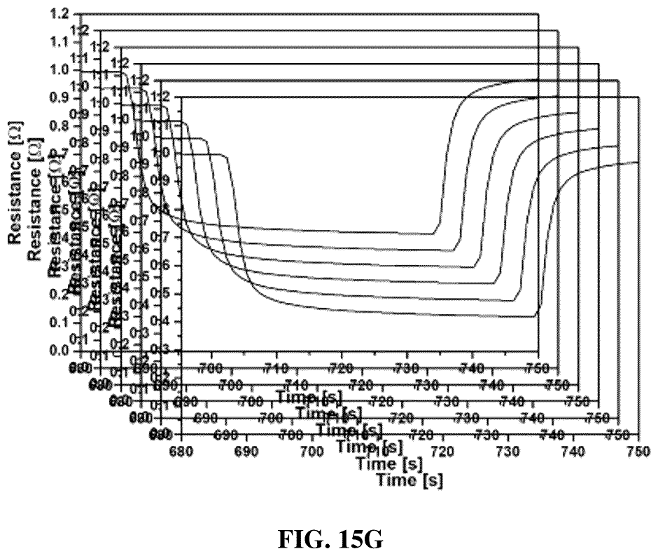

FIGS. 15A, 15B, 15C, 15D, 15E, 15F, and 15G are each a graph showing data obtained when a deep learning portion of an early diagnosis system using a multi-sensor according to a second embodiment of the disclosure performs deep learning based on different electrochemical signals, which are one-dimensional signals.

The graph shown in FIG. 15A is the total data that was measured several times continuously in a response-recovery-response-recovery manner for the analysis target gas during the experiment.

The value of the total data is the reactivity (Response_{G}), which means the value converted to the relative change rate of the value changed by the chemical reaction between the gas molecule and the sensor from the stabilized resistance after the air injection, and the equation for the reactivity (Response) is defined as [Mathematical Formula 1] below.

$$response = \frac{R - {R}_{0}}{{R}_{0}} \qquad [ Mathematical Formula 1 ]$$

(Here, R=initially measured resistance, R₀=resistance changed by the chemical reaction between the analysis target gas and the sensor)

In FIG. 15B, the deep learning portion 320′ extracts (cuts) one-time reactivity data of one cycle (=1 cycle, response-recovery) from continuous measurement data, and this deep learning portion 320′ extracts one-time reactivity data for each gas for each different electrochemical signal that reacted with each sensor.

Referring to FIG. 15B, a decrease in reactivity data indicates a change in resistance value due to chemical bonding between the sensor and the analysis target gas, while an increase in reactivity data indicates that the analysis target gas is desorbed into the air and the resistance is recovered.

Referring to FIG. 15C, the deep learning portion 320′ converts the one-time reactivity data for each gas to a reactivity value of 0-1 through a normalization process. If the value is reduced to 0-1, the amount of computation is reduced, which has an economical advantage.

Referring to FIGS. 15D, 15E, and 15F, the deep learning portion 320′ divides the normalized data into training data, validation data during training, and test data.

Referring to FIG. 15D, the training data is used to update the deep learning model by calculating the difference (loss) with the true value (True label) after one operation, and thus the deep learning model is optimized to fit the data.

Referring to FIG. 15D, the deep learning portion 320′ is repeated tens to hundreds of times according to the set epoch.

Referring to FIG. 15E, validation data during training is used during a training process, and when the deep learning model is operated and updated once, a validation evaluation (mini test) is performed so that the training status may be determined with a small number of data that is not duplicated with the training data. Therefore, it is possible to prevent overfitting, where the deep learning model is optimized only for training data.

Referring to FIG. 15F, the test data is used to evaluate the deep learning model after training is completed, and only an operation value is obtained without updating the deep learning model.

Referring to FIG. 15G, the two-dimensional data in which one-dimensional data is stacked according to nine channels is completed. The two-dimensional data above is used as a channel when input to the deep learning model.

In the conventional technology, since a two-dimensional image is used for input, image characteristics such as RGB values are used for the channel, but in our technology, different electrochemical signals obtained simultaneously for the same target material from the channel electrode 131′ of the multi-sensor 100′ are input to the deep learning model as they are in the channel of the deep learning portion 320′. This provides several grounds for determining which correct answer the data is for.

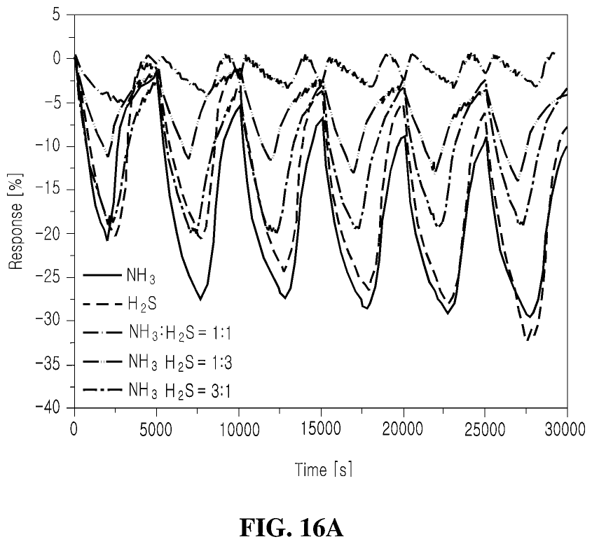

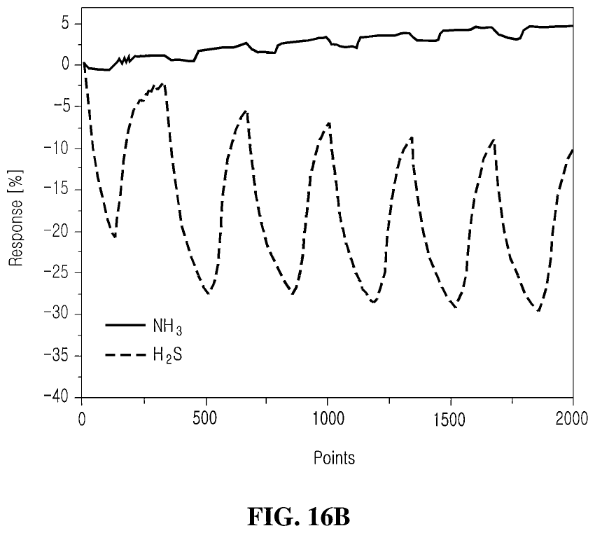

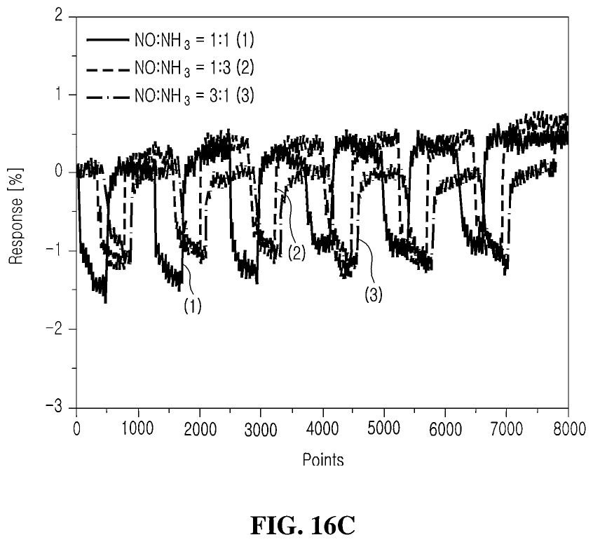

FIGS. 16A, 16B, and 16C are each a graph showing the reactivity over time of the composition ratio of an analysis target gas obtained by a multi-sensor equipped in an early diagnosis system using a multi-sensor according to a second embodiment of the disclosure.

FIGS. 16A, 16B, and 16C are graphs showing different electrochemical signals over time actually derived by a plurality of sensors when implementing an early diagnosis system using a multi-sensor according to a second embodiment of the disclosure.

Referring to FIGS. 16A, 16B, and 16C, the reactivity of a single gas is not always constant, so it is difficult to regard it as linear. Even when the reactivity is similar, the size may be reversed for each measurement, and in particular, even if the hydrogen sulfide (H₂S) content is high, the reactivity does not increase proportionally. Since the order of reactivity may sometimes change, linearity cannot be determined based on only raw data.

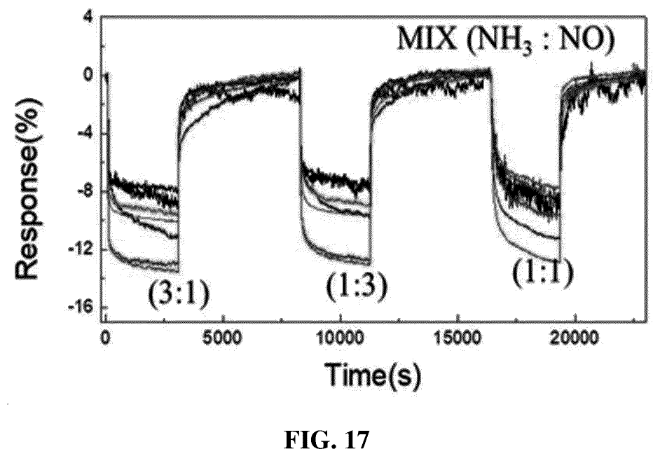

FIG. 17 is a graph showing the reactivity over time of the composition ratio of an analysis target gas in an early diagnosis system using a multi-sensor according to a second embodiment of the disclosure.

Referring to FIG. 17, the actual measurement environment is not always constant, so it is difficult to secure mathematical consistency in all environments. An analysis target gas may not show regular results due to differences in chemical bonds depending on the ratio, and the reaction to the flowing gas may generate noise depending on the measurement situation.

For this reason, it is difficult to find a consistent mathematical rule for the reaction of the material, so a multidimensional operation of an artificial neural network was utilized.

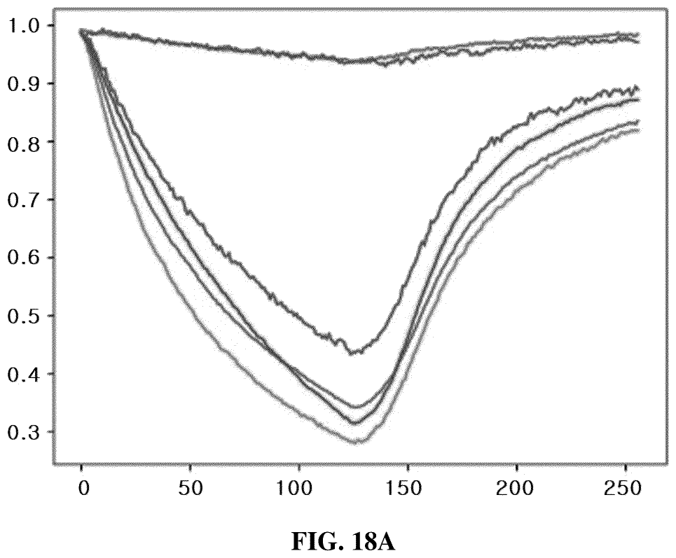

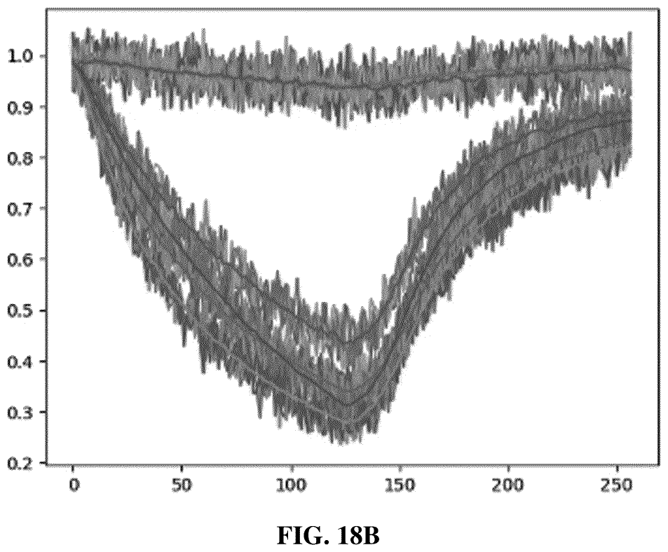

FIG. 18 ((a) and (b)) is graphs showing the before and after of the augmentation of data obtained when a deep learning portion equipped in an early diagnosis system using a multi-sensor according to a second embodiment of the disclosure performs deep learning based on different electrochemical signals, which are one-dimensional signals.

In addition, data augmentation techniques are used for training data.

Accordingly, the deep learning portion 320′ augments the training data illustrated in (a) of FIG. 18 as illustrated in (b) of FIG. 18.

For example, when there are 10 pieces of actual data, random data with noise forcibly added in the x-axis and y-axis directions may be created from the original data, thereby increasing the total data to tens to hundreds. In the case of an analysis target gas, since the detection time is long, a lot of training data may be secured with a small number of experiments, so it may also be used to prepare for noise that may occur in an actual measurement environment.

Here, since the experimental environment has little noise, data with noise is also trained to learn a plurality of cases for the same correct answer.

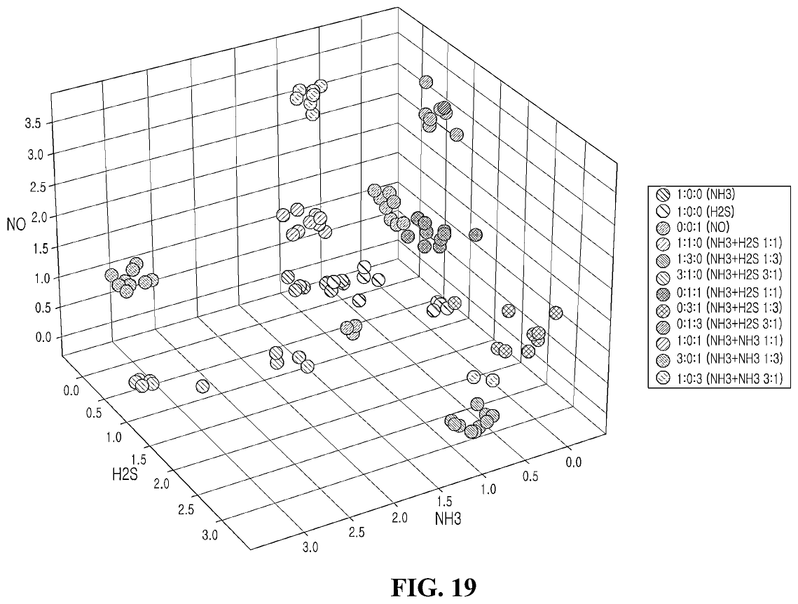

FIG. 19 is a graph showing the composition ratio of an analysis target gas obtained through deep learning in an early diagnosis system using a multi-sensor according to a second embodiment of the disclosure.

The diagnostic portion 330′ may determine the composition ratio of ammonia NH₃, hydrogen sulfide H₂O, and carbon monoxide NO based on the learning information transmitted from the deep learning portion 320′, as shown in FIG. 19.

For this purpose, the diagnostic portion 330′ may derive the composition of the analysis target gas by utilizing a pre-learned deep learning algorithm based on different electrochemical signals received from the sensor portion 120′.

The diagnostic portion 330′ may include a built-in processor and memory, and may execute software that may implement various deep learning algorithms and data processing techniques.

In addition, the diagnostic portion 330′ may visually express the analysis results through a user interface, and, if necessary, communicate with a remote server or other device to exchange data.

TABLE 2

| | chemical | | |
|---|---|---|---|
| humidity | vapor | R2 | accuracy |
| conditions | composition | score | [%] |
| low | NH₃—NO | 0.987 | 100 |
| humidity | NO₂—NO | 0.986 | 100 |
| condition | NH₃—NO₂ | 0.984 | 100 |
| high | NH₃—H₂S | 0.995 | 100 |
| humidity | H₂S—NO | 0.995 | 100 |
| condition | NH₃—NO | 0.990 | 99.18 |

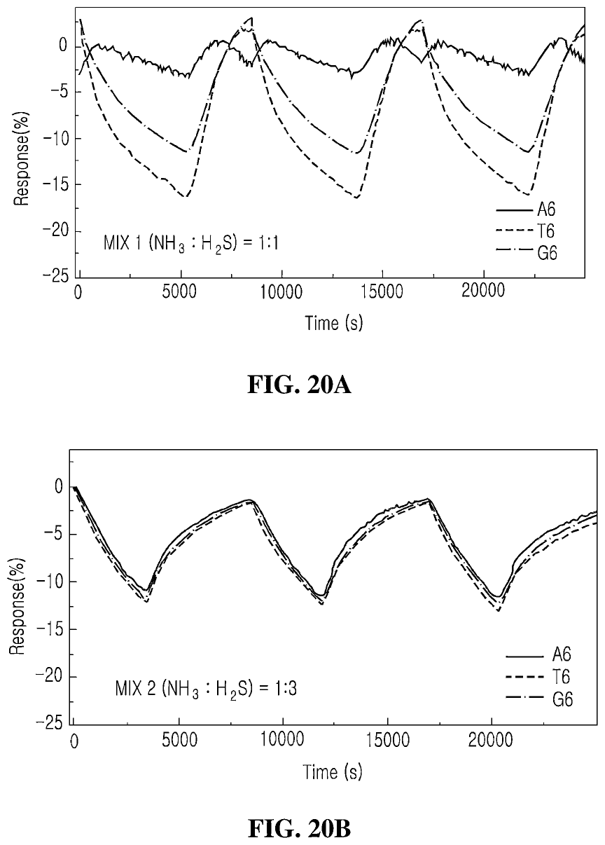

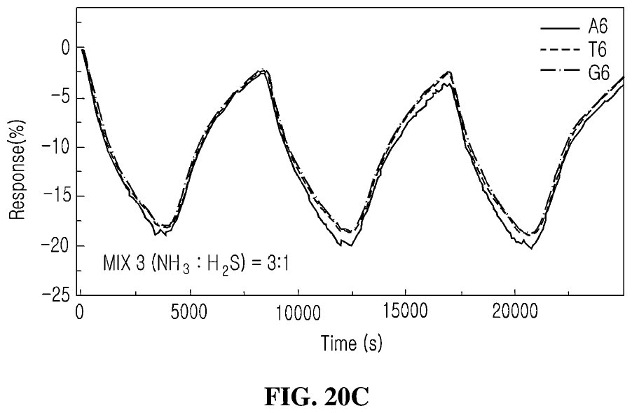

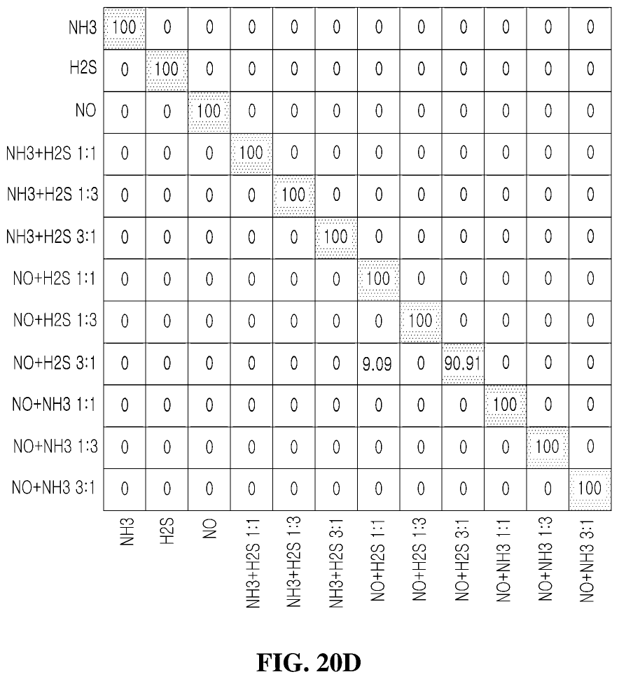

FIGS. 20A, 20B, and 20C are each a graph showing response graphs of an analysis target gas under high-humidity conditions in an early diagnosis system using a multi-sensor according to a second embodiment of the disclosure. FIG. 20D is a view showing the results of deep learning classification accuracy for the graphs of FIGS. 20A, 20B, and 20C.

Referring to FIGS. 20A, 20B, and 20C, disease biomarker gases such as ammonia (NH₃) and hydrogen sulfide (H₂S) may be seen reacting with a plurality of sensors, mimicking human breathing under conditions of 80% relative humidity.

Referring to FIG. 20D, deep learning is used to analyze gases with various mixing ratios of ammonia, hydrogen sulfide, and nitrogen monoxide, and the classification results are shown with an accuracy of over 99%.

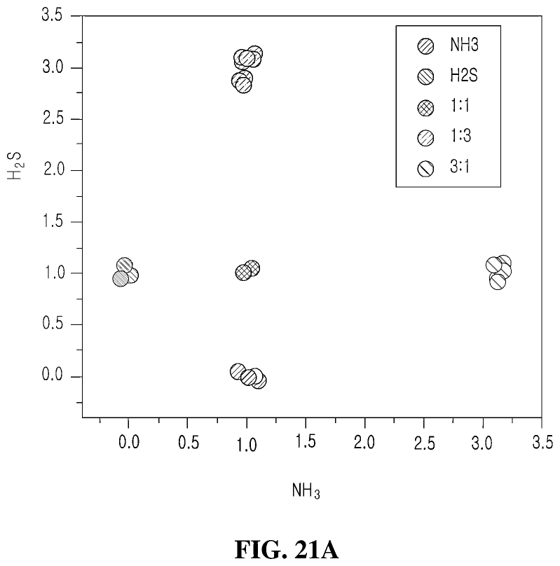

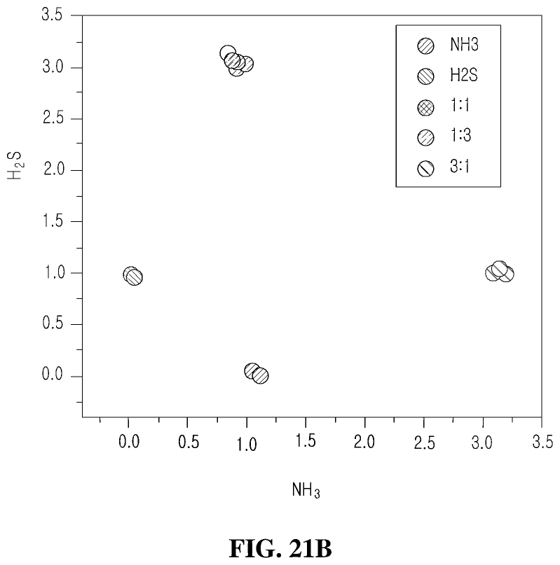

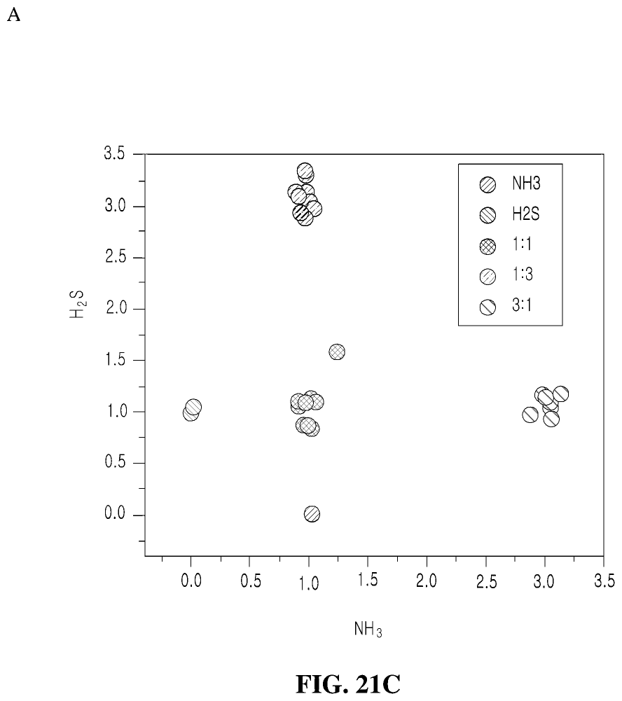

FIG. 21A is a graph predicting the composition ratio of NH₃ and H₂S by analyzing different electrochemical signals for an analysis target gas under high-humidity conditions in an early diagnosis system using a multi-sensor according to a second embodiment of the disclosure. FIG. 21B is a graph predicting the composition ratio of NO and H₂S by analyzing different electrochemical signals for an analysis target gas under high-humidity conditions using deep learning in an early diagnosis system using a multi-sensor according to a second embodiment of the disclosure. FIG. 21C is a graph predicting the composition ratio of NO and NH₃ by analyzing different electrochemical signals for an analysis target gas under high-humidity conditions using deep learning in an early diagnosis system using a multi-sensor according to a second embodiment of the disclosure.

Specifically, the diagnostic portion 330′ may obtain a composition ratio of ammonia (NH₃) and hydrogen sulfide (H₂O) as shown in FIG. 21A, obtain a composition ratio of hydrogen sulfide (H₂O) and carbon monoxide (NO) as shown in FIG. 21B, and obtain a composition ratio of ammonia (NH₃) and carbon monoxide (NO) as shown in FIG. 21C.

In FIGS. 21A, 21B, and 21C, it is possible to see the results of the deep learning-based ratio analysis of the analysis target gas. Unlike the simple classification of FIGS. 20A, 20B, and 20C, this is a prediction of the actual mixing ratio, and a user may choose between simple classification or mixing ratio prediction, and may receive a more reliable diagnosis by using simple classification and mixing ratio together.

Accordingly, the diagnostic portion 330′ determines the type and composition ratio of the analysis target gas as described above, and diagnoses one of the diseases among liver disease, kidney disease, gastric ulcer, duodenal ulcer, odor-related disease, lung cancer, and pancreatitis.

Specifically, the diagnostic portion 330′ diagnoses that a patient is suffering from liver disease, kidney disease, gastric ulcer, and duodenal ulcer when the composition ratio of ammonia is the highest among ammonia, hydrogen sulfide, and carbon monoxide.

Meanwhile, if the composition ratio of hydrogen sulfide is the highest among ammonia, hydrogen sulfide, and carbon monoxide, the diagnostic portion 330′ diagnoses as suffering from odor-related disease and pancreatitis.

On the other hand, the diagnostic portion 330′ diagnoses that a patient has lung cancer if the composition ratios of hydrogen sulfide and carbon monoxide among ammonia, hydrogen sulfide, and carbon monoxide are the same.

Furthermore, the diagnostic device 300 may transmit to the smart device 500 determination information on the type and composition ratio of the analysis target gas diagnosed in the diagnostic portion 330 and diagnosis information on diagnosing any one of liver disease, kidney disease, gastric ulcer, duodenal ulcer, odor-related disease, lung cancer, and pancreatitis, and the diagnostic device 300 for this purpose may communicate wirelessly with the smart device 500.

The smart device 500 according to this receives and displays determination information and diagnostic information transmitted from the diagnostic portion 300.

For example, the smart device 500 includes all electronic devices capable of wireless communication, including smartphones, tablet PCs, etc.

The description of the disclosure is for illustrative purposes, and those skilled in the art will understand that it can be easily modified into other specific forms without changing the technical idea or essential features of the disclosure. Therefore, the embodiments described above should be understood as being exemplary in all respects and not limiting. For example, each component described as a single type may be implemented in a distributed manner, and likewise, components described as distributed may be implemented in a combined form.

The scope of the disclosure is indicated by the following claims, and all changes or modifications derived from the meaning and scope of the claims and their equivalent concepts should be interpreted as being included in the scope of the disclosure.

### EXPLANATION OF REFERENCE NUMERALS

100, 100′: multi-sensor
110, 110′: substrate portion
111, 111′: substrate
112, 112′: silicon wafer
120, 120′: sensor portion
121, 121′: upper sensor
121a, 121a′: first upper sensor
121b, 121b′: second upper sensor
121c, 121c′: third upper sensor
122, 122′: central sensor
122a, 122a′: first central sensor
122b, 122b′: second central sensor
122c, 122c′: third central sensor
123, 123′: lower sensor
123a, 123a′: first lower sensor
123b, 123b′: second lower sensor
123c, 123c′: third lower sensor
130, 130′: electrode portion
131, 131′: electrode
132, 132′: connection electrode
140, 140′: multi-channel portion
141, 141′: body
142, 142′: upper body hole
142a, 142a′: first upper body hole
142b, 142b′: second upper body hole
142c, 142c′: third upper body hole
143, 143′: central body hole
143a, 143a: first central body hole
143b, 143b: second central body hole
143c, 143c: third central body hole
144, 144′: lower body hole
144a, 144a′: first lower body hole
144b, 144b: second lower body hole
144c, 144c′: third lower body hole
200, 200′: measuring device
300, 300′: diagnostic device
310, 310′: data storage portion
320, 320′: deep learning portion
330, 330′: diagnostic portion
400, 400′: early diagnosis system using multi-sensor

1. A multi-sensor, comprising:
a substrate portion;
a sensor portion comprising a plurality of sensors arranged in a matrix on the substrate portion;
a plurality of electrode portions arranged radially from a center of the sensor portion on the substrate portion and electrically connected to the plurality of sensors; and
a multi-channel portion positioned above the plurality of sensors where a plurality of body holes are defined for target materials to be introduced,
wherein the plurality of sensors configured to generate different electric signals for the target materials and transmit the different electric signals to the plurality of electrode portions, respectively.

2. The multi-sensor of claim 1, wherein the target materials, which are liquid protein separated from blood, comprise alpha synuclein, beta amyloid, and tau protein.

3. The multi-sensor of claim 2, wherein the different electric signals are different electrical reactive signals, and the plurality of sensors interact with the target materials to generate the different electrical reactive signals to detect a cause material of a degenerative neurological disease and transmit the different electrical reactive signals to the plurality of electrode portions, respectively.

4. The multi-sensor of claim 2, wherein the plurality of sensors are made of different graphene oxides and configured to generate the different electric signals due to changes in properties of the different graphene oxides through interactions with the target materials.

5. The multi-sensor of claim 1, wherein the target materials, which are an analysis target gas that is an animal's exhaled gas, comprise ammonia, hydrogen sulfide, and nitrogen monoxide.

6. The multi-sensor of claim 5, wherein the different electric signals are different electrochemical signals, and the plurality of sensors generate the different electrochemical signals to diagnose at least one of liver, kidney, gastric ulcer, duodenal ulcer, odor-related disease, lung cancer, and pancreatitis, and transmit the different electrochemical signals to the plurality of electrode portions, respectively.

7. The multi-sensor of claim 2, wherein the plurality of sensors include
an upper sensor positioned on an upper surface of the substrate portion and arranged in a first row,
a central sensor positioned on the upper surface of the substrate portion and arranged in a second row below the upper sensor, and
a lower sensor positioned on the upper surface of the substrate portion and arranged in a third row below the central sensor,
wherein the upper sensor is a second reduced graphene oxide,
the central sensor is a first reduced graphene oxide, and
the lower sensor is a graphene oxide.

8. The multi-sensor of claim 5, wherein an ssDNA-bound graphene comprises a first graphene functionalized with one type of ssDNA and a second graphene functionalized with two types of ssDNA,
wherein the plurality of sensors include
an upper sensor positioned on an upper surface of the substrate portion and arranged in a first row,
a central sensor positioned on the upper surface of the substrate portion and arranged in a second row below the upper sensor, and
a lower sensor positioned on the upper surface of the substrate portion and arranged in a third row below the central sensor, and
wherein the upper sensor is the first graphene functionalized with the one type of ssDNA, the central sensor is the second graphene functionalized with the two types of ssDNA, and the lower sensor is a single graphene.

9. An early diagnosis system using a multi-sensor, the early diagnosis system comprising:
the multi-sensor according to claim 1;
a measuring device electrically connected to the plurality of electrode portions and measuring the different electric signals; and
a diagnostic device configured to inputs the different electric signals transmitted from the measuring device into a deep learning model and then diagnose a disease through regression analysis,
wherein the diagnostic device comprises a deep learning portion configured to improves a diagnostic speed by only using an encoding scheme configured to increases and then decreases a number of channels where the different electrical signals, which are one-dimensional signals, are each grouped.

10. The early diagnosis system of claim 9, wherein the target materials, which are liquid protein separated from blood, comprise alpha synuclein, beta amyloid, and tau protein, and
the different electric signals are different electrical reactive signals, and
wherein the diagnostic device comprises;
a data storage portion configured to stores the different electric signals transmitted from the measuring device; and
a diagnostic portion configured to simultaneously diagnoses Alzheimer's disease and Parkinson's disease by determining a presence and a mixing state of the alpha synuclein, the beta amyloid, and the tau protein.

11. The early diagnosis system of claim 10, wherein the diagnostic portion configured to diagnose a patient who provided the target materials as having the Alzheimer's disease when a mixing ratio of the alpha synuclein, the beta amyloid, and the tau protein is 3-5:6-14:2-3.

12. The early diagnosis system of claim 10, wherein the diagnostic portion configured to diagnose a patient who provided the target materials as having the Parkinson's disease when a mixing ratio of the alpha synuclein, the beta amyloid, and the tau protein is 5-13:4-8:1-3.

13. The early diagnosis system of claim 9, wherein the different electric signals are different electrochemical signals, and
the target materials, which are an analysis target gas that is an animal's exhaled gas, comprise ammonia, hydrogen sulfide, and carbon monoxide, and
wherein the diagnostic device comprises:
a data storage portion configured to stores the different electrochemical signals transmitted from the measuring device; and
diagnostic portion configured to diagnoses at least one of liver disease, kidney disease, gastric ulcer, duodenal ulcer, odor-related disease, lung cancer, and pancreatitis by determining a type and a composition ratio of the analysis target gas.

14. The early diagnosis system of claim 13, wherein the diagnostic portion configured to diagnoses the liver disease, kidney disease, gastric ulcer and duodenal ulcer when the ammonia is present in higher amounts than hydrogen sulfide and carbon monoxide.

15. The early diagnosis system of claim 13, wherein the diagnostic portion configured to diagnose the odor-related disease and pancreatitis when the hydrogen sulfide is present in higher amounts than the ammonia and carbon monoxide.

16. The early diagnosis system of claim 13, wherein the diagnostic portion configured to diagnoses lung cancer when the hydrogen sulfide and the carbon monoxide are in equal amounts among the ammonia, the hydrogen sulfide and the carbon monoxide.
